(19) United States
(12) Patent Application Publication
(10) Pub. No.: US 2017/0224730 A1
(43) Pub. Date: Aug. 10, 2017

(54) ANTI-CANCER EFFECTS OF PROTEASOME INHIBITORS IN COMBINATION WITH GLUCOCORTICOIDS, ARSENIC CONTAINING COMPOUNDS, AND ASCORBIC ACID

(71) Applicant: Institute for Myeloma & Bone Cancer Research, West Hollywood, CA (US)

(72) Inventor: James R. BERENSON, West Hollywood, CA (US)

(73) Assignee: Institute for Myeloma & Bone Cancer Research, West Hollywood, CA (US)

(21) Appl. No.: 15/317,690

(22) PCT Filed: Jun. 10, 2015
(86) PCT No.: PCT/US2015/035023

§ 371 Date: Dec. 9, 2016

Related U.S. Application Data
(60) Provisional application No. 62/010,391, filed on Jun. 10, 2014.

(51) Int. Cl. A61K 33/36; A61K 9/00; A61K 31/375; A61K 38/07; A61K 31/573

(52) U.S. Cl. CPC A61K 33/36; A61K 38/07; A61K 31/573; A61K 31/375; A61K 9/0019; A61K 9/0053

(57) ABSTRACT

The present invention provides methods of treatment for hematological malignancies involving synergistic combination of a proteasome inhibitor, a glucocorticoid, an arsenic-containing compound, and ascorbic acid or a derivative thereof provide an unexpected efficacy in the treatment for hematological disorders. The hematological disorders treated by the current invention include multiple myeloma, and may also include hematological disorders that are refractory to prior cancer treatments, or relapsed hematologic disorders.

### CROSS REFERENCE TO RELATED APPLICATIONS

This application claims the benefit under 35 U.S.C. §119(e) of U.S. Provisional Application No. 62/010,391, filed Jun. 10, 2014, which is incorporated by reference herein in its entirety.

### BACKGROUND

### Technical Field

The invention generally relates to novel methods of treating hematological malignancies, including multiple myeloma. More particularly the invention relates to methods of treating hematological malignancies with a proteasome inhibitor, glucocorticoid, arsenic containing compound, and ascorbic acid or a derivative thereof.

Description of the Related Art

Multiple myeloma is a malignancy characterized by the expansion of plasma cells that produce monoclonal immunoglobulin (IgG, IgA, IgD, IgE, or free λ or κ light chains). The overall survival of patients with multiple myeloma varies greatly from a few months to many years; the mean is approximately five years. Anemia, hypercalcemia and bone lesions correlate directly with total mass of myeloma cells and have important prognostic significance. Other prognostic factors include age, the plasma cell labeling index, serum albumin, β2-microglobulin, C-reactive protein, thymidine kinase, and soluble interleukin 6 receptor. Major complications, such as infection and renal insufficiency, are the main causes of death for myeloma patients.

Current therapies for multiple myeloma fail to cure the disease and nearly all patients eventually develop resistance to these therapies. Moreover, there are a paucity of multiple myeloma targets and a lack of therapeutic options that are effective in overcoming drug resistance.

### BRIEF SUMMARY

The present invention generally provides novel and synergistic treatments for multiple myeloma and related B cell disorders.

In various embodiments, a method of treating or preventing a hematological malignancy in a subject comprising administering to the subject a proteasome inhibitor, a glucocorticoid, an arsenic-containing compound, and ascorbic acid or a derivative thereof is provided.

In particular embodiments, the hematological malignancy is selected from the group consisting of: multiple myeloma, chronic lymphocytic leukemia, or B-cell non-Hodgkin lymphoma.

In additional embodiments, the proteasome inhibitor is selected from the group consisting of: bortezomib, carfilzomib, oprozomib, ixazomib citrate, marizomib, delanzomib, and syringolin A.

In some embodiments, the proteasome inhibitor is carfilzomib.

In particular embodiments, the glucocorticoid is selected from the group consisting of: hydroxycortisone, cortisone, desoxycorticosterone, fludrocortisone, betamethasome, dexamethasone, prednisolone, prednisone, methylprednisolone, methylprednisone, paramethasone, triamcinolone, flumethasone, fluocinolone, fluocinonide, fluprednisolone, halcinonide, flurandrenolide, meprednisone, and medrysone.

In particular embodiments, the glucocorticoid is dexamethasone.

In certain embodiments, the arsenic-containing compound is selected from the group consisting of: arsenic trioxide (As2O3), arsenic pentoxide (As2O5), arsenic hexoxide As4O6), arsenic triselenide (As2Se3), arsenic disulfide (As2S2), arsenic trisulfide (As2S3), arsenic pentasulfide (As2O5), arsenic tritelluride (As2Te3), sodium arsenate (Na2HAsO4), potassium arsenate (KH2AsO4), and sodium arsenyl tartrate (NaC4H4AsO6).

In further embodiments, the arsenic-containing compound is arsenic trioxide.

In additional embodiments, the ascorbic acid or derivative thereof is selected from the group consisting of: ascorbic acid, L-ascorbic acid-2-pyrophosphate esters, L-ascorbic acid-2-triphosphate esters, L-ascorbic acid-2-polyphosphate esters, sodium L-ascorbic acid-2-phosphate-6-palmitate, L-ascorbic acid-2-phosphate-6-palmitate, L-ascorbic acid-2-phosphate-6-stearate, L-ascorbic acid-2-phosphate-6-oleate and L-ascorbic acid-2-phosphate-6-arachidonate, 5,6-0-isoalkylidene ascorbic acid, 5,6-0-isopropylidine ascorbic acid, and L-ascorbate 2-sulphate.

In particular embodiments, the ascorbic acid or derivative thereof is ascorbic acid.

In further embodiments, the methods comprise administering carfilzomib, arsenic trioxide, dexamethasone, and ascorbic acid.

In certain embodiments, the methods comprise administering carfilzomib intravenously.

In particular embodiments, the methods comprise administering carfilzomib at a dose of 1-100 mg/m².

In some embodiments, the methods comprise administering arsenic trioxide intravenously.

In additional embodiments, the methods comprise administering arsenic trioxide at a dose of 0-5 mg/kg.

In particular embodiments, the methods comprise administering dexamethasone orally.

In further embodiments, the methods comprise administering dexamethasone intravenously.

In particular embodiments, the methods comprise administering dexamethasone at a dose of 1-100 mg.

In certain embodiments, the methods comprise administering ascorbic acid orally.

In further embodiments, the methods comprise administering ascorbic acid orally at a dose of 100-2000 mg.

In some embodiments, the methods comprise administering ascorbic acid intravenously.

In particular embodiments, the methods comprise administering ascorbic acid intravenously at a dose of 1-50 mg.

In various embodiments, a method of treating or preventing multiple myeloma in a subject comprising administering to the subject a proteasome inhibitor, arsenic trioxide, a glucocorticoid, and ascorbic acid is provided.

In certain embodiments, the proteasome inhibitor is selected from the group consisting of: wherein the proteasome inhibitor is selected from the group consisting of: bortezomib, carfilzomib, oprozomib, ixazomib citrate, marizomib, delanzomib, and syringolin A.

In some embodiments, the proteasome inhibitor is carfilzomib.

In some embodiments, the glucocorticoid is selected from the group consisting of: hydroxycortisone, cortisone, desoxycorticosterone, fludrocortisone, betamethasome, dexamethasone, prednisolone, prednisone, methylprednisolone, methylprednisone, paramethasone, triamcinolone, flumethasone, fluocinolone, fluocinonide, fluprednisolone, halcinonide, flurandrenolide, meprednisone, and medrysone.

In particular embodiments, the glucocorticoid is dexamethasone.

In additional embodiments, the arsenic-containing compound is selected from the group consisting of: arsenic trioxide (As2O3), arsenic pentoxide (As2O5), arsenic hexoxide As4O6), arsenic triselenide (As2Se3), arsenic disulfide (As2S2), arsenic trisulfide (As2S3), arsenic pentasulfide (As2O5), arsenic tritelluride (As2Te3), sodium arsenate (Na2HAsO4), potassium arsenate (KH2AsO4), and sodium arsenyl tartrate (NaC4H4AsO6).

In particular embodiments, the arsenic-containing compound is arsenic trioxide.

In some embodiments, the ascorbic acid or derivative thereof is selected from the group consisting of: ascorbic acid, L-ascorbic acid-2-pyrophosphate esters, L-ascorbic acid-2-triphosphate esters, L-ascorbic acid-2-polyphosphate esters, sodium L-ascorbic acid-2-phosphate-6-palmitate, L-ascorbic acid-2-phosphate-6-palmitate, L-ascorbic acid-2-phosphate-6-stearate, L-ascorbic acid-2-phosphate-6-oleate and L-ascorbic acid-2-phosphate-6-arachidonate, 5,6-0-isoalkylidene ascorbic acid, 5,6-0-isopropylidine ascorbic acid, and L-ascorbate 2-sulphate.

In further embodiments, the ascorbic acid or derivative thereof is ascorbic acid.

In additional embodiments, the methods comprise administering carfilzomib, arsenic trioxide, dexamethasone, and ascorbic acid.

In particular embodiments, the methods comprise administering carfilzomib intravenously.

In particular embodiments, the methods comprise administering carfilzomib at a dose of 1-100 mg/m².

In further embodiments, the methods comprise administering arsenic trioxide intravenously.

In certain embodiments, the methods comprise administering arsenic trioxide at a dose of 0-5 mg/kg.

In certain embodiments, the methods comprise administering dexamethasone orally.

In some embodiments, the methods comprise administering dexamethasone intravenously.

In particular embodiments, the methods comprise administering dexamethasone at a dose of 1-100 mg/m².

In further embodiments, the methods comprise administering ascorbic acid orally.

In additional embodiments, the methods comprise administering ascorbic acid orally at a dose of 100-2000 mg/m².

In particular embodiments, the methods comprise administering ascorbic acid intravenously.

In some embodiments, the methods comprise administering ascorbic acid intravenously at a dose of 1-50 mg/m².

In various embodiments, a method of treating or preventing a relapsed hematological malignancy in a subject comprising administering to the subject, a glucocorticoid, an arsenic-containing compound, and ascorbic acid or a derivative thereof is provided.

In various other embodiments, a method of treating or preventing hematological malignancy that is refractory to a prior treatment or treatments for cancer in a subject comprising administering to the subject a proteasome inhibitor, a glucocorticoid, an arsenic-containing compound, and ascorbic acid or a derivative thereof is provided.

In further embodiments, the hematological malignancy is selected from the group consisting of: multiple myeloma, chronic lymphocytic leukemia, or B-cell non-Hodgkin lymphoma.

In particular embodiments, the subject was previously treated with a proteasome inhibitor.

In additional embodiments, the subject was previously treated with carfilzomib.

In additional embodiments, the subject was previously treated with a chemotherapeutic agent.

In some embodiments, the subject was previously treated with a glucocorticoid.

In particular embodiments, the subject was previously treated with ascorbic acid.

In certain embodiments, the proteasome inhibitor is selected from the group consisting of: wherein the proteasome inhibitor is selected from the group consisting of: bortezomib, carfilzomib, oprozomib, ixazomib citrate, marizomib, delanzomib, and syringolin A.

In further embodiments, the proteasome inhibitor is carfilzomib.

In further embodiments, the glucocorticoid is selected from the group consisting of: hydroxycortisone, cortisone, desoxycorticosterone, fludrocortisone, betamethasome, dexamethasone, prednisolone, prednisone, methylprednisolone, methylprednisone, paramethasone, triamcinolone, flumethasone, fluocinolone, fluocinonide, fluprednisolone, halcinonide, flurandrenolide, meprednisone, and medrysone.

In particular embodiments, the glucocorticoid is dexamethasone.

In some embodiments, the arsenic-containing compound is selected from the group consisting of: arsenic trioxide (As2O3), arsenic pentoxide (As2O5), arsenic hexoxide As4O6), arsenic triselenide (As2Se3), arsenic disulfide (As2S2), arsenic trisulfide (As2S3), arsenic pentasulfide (As2O5), arsenic tritelluride (As2Te3), sodium arsenate (Na2HAsO4), potassium arsenate (KH2AsO4), and sodium arsenyl tartrate (NaC4H4AsO6).

In certain embodiments, the arsenic containing compound is arsenic trioxide.

In further embodiments, the ascorbic acid or derivative thereof is selected from the group consisting of: ascorbic acid, L-ascorbic acid-2-pyrophosphate esters, L-ascorbic acid-2-triphosphate esters, L-ascorbic acid-2-polyphosphate esters, sodium L-ascorbic acid-2-phosphate-6-palmitate, L-ascorbic acid-2-phosphate-6-palmitate, L-ascorbic acid-2-phosphate-6-stearate, L-ascorbic acid-2-phosphate-6-oleate and L-ascorbic acid-2-phosphate-6-arachidonate, 5,6-0-isoalkylidene ascorbic acid, 5,6-0-isopropylidine ascorbic acid, and L-ascorbate 2-sulphate.

In certain embodiments, the ascorbic acid or derivative thereof is ascorbic acid.

In particular embodiments, the methods comprise administering carfilzomib, arsenic trioxide, dexamethasone, and ascorbic acid.

In particular embodiments, the methods comprise administering carfilzomib intravenously.

In additional embodiments, the methods comprise administering carfilzomib at a dose of 1-100 mg/m².

In further embodiments, the methods comprise administering arsenic trioxide intravenously.

In some embodiments, the methods comprise administering arsenic trioxide at a dose of 0-5 mg/kg.

In particular embodiments, the methods comprise administering dexamethasone orally.

In certain embodiments, the methods comprise administering dexamethasone intravenously.

In particular embodiments, the methods comprise administering dexamethasone at a dose of 1-100 mg/m².

In further embodiments, the methods comprise administering ascorbic acid orally.

In particular embodiments, the methods comprise administering ascorbic acid orally at a dose of 100-2000 mg/m².

In additional embodiments, the methods comprise administering ascorbic acid intravenously.

In certain embodiments, the methods comprise administering ascorbic acid intravenously at a dose of 1-50 mg/m².

### DETAILED DESCRIPTION

### A. Introduction

Multiple myeloma is an incurable disease. Alkylating agents such as melphalan and cyclophosphamide and other drugs, including vinca alkaloids, nitrosoureas and anthracyclines, are only marginally effective in treating multiple myeloma when combined with steroids and do not improve survival compared to the combination of oral melphalan and prednisone. Nor has the commonly used combination of vincristine, doxorubicin and dexamethasone improved median survival compared to other treatment regimens in randomized clinical trials.

Proteasome inhibitors (PIs) such as bortezomib or carfilzomib, the immunomodulatory agents thalidomide, pomalidomide or lenalidomide, pegylated liposomal doxorubicin, and arsenic trioxide reduce myeloma cell growth in laboratory studies and have been used clinically to treat multiple myeloma patients. High-dose chemotherapy followed by autologous hematopoietic support also represents another treatment option for multiple myeloma patients. Despite these recent improvements in multiple myeloma therapies, nearly all patients develop resistance to these therapies and eventually succumb to the disease.

In various embodiments, the present invention contemplates, in part, additional tumor targets and therapeutic options that are capable of overcoming drug resistance to hematological malignancies and improving the outcome for these patients. Surprisingly, the present inventors have discovered that using proteasome inhibitors in combination with glucocorticoids, arsenic containing compounds, and/or ascorbic acid or derivatives thereof provides a synergistic anti-cancer effect and may be used to treat patients with hematological malignancies and improve the clinical outcome. In some embodiments, the combination of these agents can be used to treat subjects refractory to previous treatments for hematological malignancies, including patients refractory to treatments with one or more of, but not all of these agents. In particular embodiments, methods to prevent, treat, or ameliorate at least one symptom of a hematological malignancy in a subject are provided.

In certain embodiments, subjects in need of treatment for a hematological malignancy including, but not limited to, chronic lymphocytic leukemias, B-cell non-Hodgkin lymphomas, and multiple myeloma are provided.

In various embodiments, a subject is administered a proteasome inhibitor, a glucocorticoid, an arsenic containing compound, and ascorbic acid or a derivative thereof.

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

The articles “a,” “an,” and “the” are used herein to refer to one or to more than one (i.e., to at least one) of the grammatical object of the article. By way of example, “an element” means one element or more than one element.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2, or 1% to a reference quantity, level, value, number, frequency, percentage, dimension, size, amount, weight or length. In particular embodiments, the terms “about” or “approximately” when preceding a numerical value indicate the value plus or minus a range of 15%, 10%, 5%, or 1%.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any treatment of a disease in a mammal, and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest.

As used herein, the phrase “ameliorating at least one symptom of” refers to decreasing one or more symptoms of the disease or condition for which the subject is being treated. In particular embodiments, the disease or condition being treated is a hematological malignancy, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function). In particular embodiments, the disease or condition being treated is a multiple myeloma, wherein the one or more symptoms ameliorated include bone pain.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also include reducing the intensity, effect, symptoms and/or burden of a disease or condition prior to onset or recurrence of the disease or condition.

As used herein, the term “amount” refers to “an amount effective” or “an effective amount” of cells sufficient to achieve a beneficial or desired prophylactic or therapeutic result, including clinical results. In one embodiment an effect amount refers to the amount of a proteasome inhibitor, a glucocorticoid, an arsenic-containing compound, and/or ascorbic acid or a derivative thereof sufficient to prevent, ameliorate one symptom of, or treat a disease, e.g., a hematological malignancy contemplated herein.

A “prophylactically effective amount” refers to an amount of a proteasome inhibitor, a glucocorticoid, an arsenic-containing compound, and/or ascorbic acid or a derivative thereof effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a proteasome inhibitor, a glucocorticoid, an arsenic-containing compound, and/or ascorbic acid or a derivative thereof may vary according to factors such as the disease state, age, sex, and weight of the individual, and the agent to elicit a desired response in the individual. A therapeutically effective amount is also one in which any toxic or detrimental effects of the agent are outweighed by the therapeutically beneficial effects. The term “therapeutically effective amount” includes an amount that is effective to “treat” a subject (e.g., a patient).

As used herein, the terms “conditions sufficient,” or “under conditions sufficient,” refer to the conditions for treating the subject, with one or more agents or compositions contemplated herein. In one embodiment, “conditions sufficient” include administering a sufficient amount, e.g., an effective amount of a proteasome inhibitor, a glucocorticoid, an arsenic containing compound, and/or ascorbic acid or a derivative thereof to a subject in need thereof.

As used herein, the terms “promoting,” “enhancing,” “stimulating,” or “increasing” generally refer to the ability of compositions contemplated herein to produce or cause a greater physiological response (i.e., measurable downstream effect), as compared to the response caused by either vehicle or a control molecule/composition. One such measurable physiological response includes, without limitation, increased cell killing and/or tumor reduction, increased survival, increased treatment efficacy compared to normal, untreated, or control-treated subjects. The physiological response may be increased by at least 5%, 10%, 20%, 30%, 40%, 50%, 60%, 70%, 80%, 90%, 100%, 125%, 150%, 175%, 200%, or greater compared to the response measured in normal, untreated, or control-treated subjects. An “increased” or “enhanced” response or property is typically “statistically significant”, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) that produced by normal, untreated, or control-treated subjects.

As used herein, the terms “decrease” or “lower,” or “lessen,” or “reduce,” or “abate” refers generally to the ability of compositions contemplated to produce or cause a lesser physiological response (i.e., downstream effects), as compared to the response caused by either vehicle or a control molecule/composition, e.g., decreased tumor volume. A “decrease” or “reduced” response is typically a “statistically significant” response, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by normal, untreated, or control-treated subject.

“Hematological malignancy” is a type of cancer that affects blood, bone marrow or lymph nodes. Hematological malignancies may derive from either of the two major blood cell lineages: myeloid and lymphoid cell lines. The myeloid cell line normally produces granulocytes, erythrocytes, thrombocytes, macrophages, and mast cells, whereas the lymphoid cell lines produce B-cells, T-cells, natural killer cells, and plasma cells. Lymphomas, lymphocytic leukemias and myeloma are from the lymphoid cell line. Illustrative examples of hematological malignancies that can be treated with compositions contemplated herein include, myelomas, leukemias, and lymphomas.

Other illustrative examples of hematological malignancies that are suitable for treatment in particular embodiments of the methods contemplated herein include, but are not limited to, multiple myeloma, acute myeloid leukemia (AML), chronic myeloid leukemia (CML), acute lymphatic leukemia (ALL), chronic lymphatic leukemia (CLL), Hodgkin's disease, Non-Hodgkin lymphoma, myelodysplastic syndrome (MDS) or myeloproliferative diseases. Usually, hematological malignancies do not form solid tumors.

A “subject,” “subject in need of treatment,” “subject in need thereof,” “individual,” or “patient” as used herein, includes any animal that exhibits a symptom of a disease, disorder, or condition that can be treated with compositions contemplated herein. In particular embodiments, the disease, disorder, or condition relates to a hematological malignancy, e.g, multiple myeloma. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in preferred embodiments, the subject is a human.

The term “relapse” refers to the diagnosis of return, or signs and symptoms of return, of a cancer after a period of improvement or remission.

“Remission,” also known as “clinical remission,” includes both partial and complete remission. In partial remission, some, but not all, signs and symptoms of cancer have disappeared. In complete remission, all signs and symptoms of cancer have disappeared, although cancer still may be in the body.

“Refractory” refers to a cancer that is resistant to, or non-responsive to, therapy with a particular therapeutic agent. A cancer can be refractory from the onset of treatment (i.e., non-responsive to initial exposure to the therapeutic agent), or as a result of developing resistance to the therapeutic agent, either over the course of a first treatment period or during a subsequent treatment period.

The term “agent” refers to a natural or synthetic polypeptide, polynucleotide, carbohydrate, fatty acid, chemical compound, or small organic molecule.

The term “small molecule” encompasses numerous biological and chemical classes, including synthetic, semi-synthetic, or naturally-occurring inorganic or organic molecules, including synthetic, recombinant or naturally-occurring compounds. A “small molecule” also refers to an agent that has a molecular weight of less than about 5 kD, less than about 4 kD, less than about 3 kD, less than about 2 kD, less than about 1 kD, or less than about .SkD. Small molecules include, but are not limited to: nucleic acids, peptidomimetics, peptoids, carbohydrates, lipids or other organic or inorganic molecules. In particular embodiments, small molecules are obtained from a combinatorial small organic molecule or peptide library containing a large number of potential therapeutic compounds. Such “combinatorial chemical libraries” or “ligand libraries” can be screened separately or screened in pools, to identify those library members particular chemical species or subclasses that display the desired characteristic activity of inhibiting proteasome activity, possessing similar properties to arsenic containing compounds or ascorbic acid, or activating steroid receptors.

“M-protein”, also known as paraprotein, is an immunoglobulin or immunoglobulin light-chain that is produced in excess by the clonal proliferation of monoclonal plasma cells. Detection of paraproteins in the urine or blood can be associated with multiple myeloma.

“Immunoglobulin isotypes” refer to genetic variations or differences in the constant regions of the heavy and light chains. In humans, there are five heavy chain isotypes; IgA, IgD, IgG, IgE, and IgM; and two light chains: λ, or κ light chains.

“Platelets” are small cells found in blood. Platelets are cytosolic fragments that do not contain nuclei that are generated from megakaryocytes of the bone marrow. Platelets function, along with coagulation factors, to stop or prevent bleeding. In hematological malignancies, such as multiple myeloma, platelets levels are often reduced.

“Hematocrit”, also known in the art as “packed cell volume” or “erythrocyte volume fraction” is the ratio of the total volume of the red cells (erythrocytes) of a given sample of whole blood to the total volume of that sample, normally expressed as a percentage. This index is useful in diagnostic studies and treatment of diseases whose symptoms and manifestations may include physical changes in the blood. The measurement depends on the number and size of the erythrocytes.

“Hemoglobin” is a protein that functions physiologically as the principal carrier of oxygen in whole blood from the lungs to other body tissues. It is also the protein found in highest concentration in whole blood (normally 12-18 percent). Lower than normal values are symptomatic of anemia, which is commonly associated with multiple myeloma. The determination of hemoglobin content of whole blood is done routinely and, thus, is one of the most frequently performed clinical laboratory tests.

A “treatment cycle” as used herein refers to a course of treatment that is repeated on a regular schedule. A treatment cycle can comprise several days of treatment followed by several days of rest. For example, an agent may be administered daily for three weeks, followed by a week of no treatment, in a 28 day treatment cycle.

### C. Compositions

In various embodiments, a subject is administered one or more of the compositions contemplated herein to prevent, treat, or ameliorate at least one symptom of, a hematological malignancy. In particular embodiments, a subject is administered one or more compositions comprising a proteasome inhibitor, a glucocorticoid, an arsenic-containing compound, and/or ascorbic acid or a derivative thereof. In various embodiments, the compositions contemplated herein comprise one or more proteasome inhibitors, glucocorticoids, arsenic-containing compounds, and ascorbic acid or derivatives thereof that can be administered separately or in any suitable combination that are administered to prevent, ameliorate at least one symptom of, or treat a hematological malignancy.

The present inventors have discovered that proteasome inhibitors, when combined with glucocorticoids, arsenic-containing compounds, and ascorbic acid or derivatives thereof, are unexpectedly and synergistically effective at treating hematological malignancies. Without being bound to any particular theory, the present invention contemplates, in part, that the proteasome inhibitors, glucocorticoids, arsenic-containing compounds, and ascorbic acid or derivatives thereof are more effective in combination for treating a hematological malignancy in a subject than treatment with either of proteasome inhibitors, glucocorticoids, arsenic-containing compounds, or ascorbic acid or its derivatives thereof alone.

According to some embodiments, proteasome inhibitors of the invention are useful for treating cancer when combined with a glucocorticoid, an arsenic-containing compound, and ascorbic acid or a derivative thereof, and administered to a subject diagnosed with hematological malignancies. In some embodiments, the hematological malignancies can include, but are not limited to multiple myeloma, chronic lymphocytic leukemia, or B-cell non-Hodgkin lymphoma. In some embodiments, the hematological malignancy is refractory to previous treatments for cancer. These previous treatments may include, but are not limited to, chemotherapeutic agents. In some embodiments, the hematological malignancy is refractory to previous treatments of one or more, but not all, of a proteasome inhibitor, a glucocorticoid, an arsenic-containing compound, and ascorbic acid or a derivative thereof. In some embodiments, the hematological malignancy is refractory to different treatments that separately comprised proteasome inhibitors, glucocorticoids, arsenic-containing compounds, and ascorbic acid or derivatives thereof. In certain embodiments, a subject has not been previously treated with, and the hematological malignancy is not refractory to, a combined treatment of a proteasome inhibitor, a glucocorticoid, an arsenic-containing compound, and ascorbic acid or a derivative thereof. In particular embodiments, the hematological malignancy is relapsed.

In particular embodiments, compositions (i.e., medicaments) contemplated herein comprise one or more of a proteasome inhibitor, a glucocorticoid, an arsenic-containing compound, and ascorbic acid or a derivative thereof. In certain embodiments, the one or more of proteasome inhibitor, a glucocorticoid, an arsenic-containing compound, and ascorbic acid or a derivative thereof may be delivered in the form of a prodrug, solvate, stereoisomer, racemate, tautomer or pharmaceutically-acceptable salt thereof.

As used herein, the term “prodrug” refers to a compound that may be converted under physiological conditions or by solvolysis to a biologically active compound contemplated herein. In one embodiment, the term “prodrug” refers to a pharmaceutically acceptable metabolic precursor of a compound contemplated herein. A prodrug may be inactive when administered, but converted in vivo to an active compound. Prodrugs are typically rapidly transformed in vivo to yield a proteasome inhibitor, a glucocorticoid, an arsenic containing compound, or ascorbic acid or a derivative thereof, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987. In certain embodiments, the term “prodrug” also refers to any covalently bonded carriers, which release the active compound of the invention in vivo when such prodrug is administered to a mammalian subject. Prodrugs contemplated herein may be prepared by modifying functional groups present in the compound in such a way that the modifications are cleaved, either in routine manipulation or in vivo, to the parent compound. Prodrugs may comprise compounds wherein a hydroxy, amino or mercapto group is bonded to any group that, when the prodrug is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds and the like.

In particular embodiments, the compounds contemplated herein may be isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹F, ³²F, ³⁵S, ¹⁸F, ³⁶Cl, ¹²³I, and ¹²⁵I respectively.

### D. Proteasome Inhibitors

In eukaryotic cells, the ubiquitin-proteasome pathway is the central pathway for selective protein degradation of intracellular proteins. Proteins are targeted for proteolysis by the attachment of a polyubiquitin chain, and then rapidly degraded to small peptides by the proteasome. The proteolytic pathway is dependent upon the activities of the both the ubiquitin-conjugating system and the 26S proteasome. The 26S proteasome is a large, approximately 1500 to 2000 kDa, multi-subunit complex present in the nucleus and cytoplasm of eukaryotes. The catalytic core of this complex, referred to as the 20S proteasome, is a cylindrical structure consisting of four heptameric rings containing α- and β-subunits. The ability to recognize and bind polyubiquinated substrates is conferred by 19S subunits, which bind to each end of the 20S proteasome. These accessory subunits unfold substrates and feed them into the 20S catalytic complex while removing the attached ubiquitin molecules.

Proteasome inhibition has emerged as a novel anti-cancer therapy. The ubiquitin-proteasome pathway regulates a number of important cellular processes critical for maintaining cell homeostasis and tissue physiology including cell cycle progression, apoptosis, inflammation, cell adhesion, migration, transcription and angiogenesis. In vitro studies have demonstrated that proteasome inhibitors trigger apoptosis in a variety of tumor-derived cell lines and patient-derived cells, many of which are hematopoietic in origin, including monoblasts, T-cell and lymphocytic leukemia, promyelocytic leukemia, lymphoma, and multiple myeloma cells.

Proteasome inhibitors can induce cell cycle arrest and subsequent apoptosis in these cells.

Proteasome inhibitors are currently approved treat hematological malignancies. For example, the proteasome inhibitor bortezomib is approved in the U.S. for treatment of multiple myeloma and mantle cell lymphoma. Bortezomib was approved for third line-treatment of multiple myeloma in 2003, and later approved for first-line treatment in 2008. Another proteasome inhibitor recently approved for treatment of multiple myeloma is carfilzomib, which was approved in 2012. Yet despite the successes of these treatments, most multiple myeloma patients eventually develop resistance to proteasome inhibitors.

The term “proteasome inhibitor” refers to any substance which directly or indirectly inhibits the 20S and/or 26S proteasome or an activity thereof. In particular embodiments, proteasome inhibition is specific, i.e., the proteasome inhibitor inhibits proteasome activity at a concentration that is lower than the concentration of the inhibitor required to produce another, unrelated biological effect.

In various embodiments, a subject is administered a proteasome inhibitor, optionally in combination with glucocorticoid, an arsenic-containing compound, and ascorbic acid or a derivative thereof. Without being bound to any particular theory, the present invention contemplates, in part, that proteasome inhibitors combined with glucocorticoids, arsenic-containing compounds, and ascorbic acid or a derivative thereof, synergistically treat a hematological malignancy compared to either proteasome inhibitors, glucocorticoids, arsenic-containing compounds, or ascorbic acid or derivatives thereof alone.

In particular embodiments, a proteasome inhibitor suitable for use in the compositions and methods contemplated herein includes any agent that directly or indirectly inhibits the 20S and/or 26S proteasome or an activity thereof. In certain embodiments, a proteasome inhibitor may decrease proteasome activity in a cell about 5%, about 10%, about 15%, about 20%, about 25%, about 30%, about 20%, about 25%, about 30%, about 35%, about 40%, about 45%, about 50%, about 55%, about 60%, about 65%, about 70%, about 75%, about 80%, about 85%, about 90%, about 95%, about 100%, compared to a cell that has not been contacted by the proteasome inhibitor.

In particular embodiments, a proteasome inhibitor may decrease levels of the proteasome or a proteasome subunit in a cell about 5%, about 10%, about 15%, about 20%, about 25%, about 30%, about 20%, about 25%, about 30%, about 35%, about 40%, about 45%, about 50%, about 55%, about 60%, about 65%, about 70%, about 75%, about 80%, about 85%, about 90%, about 95%, about 100%, compared to the level of proteasome or proteasome subunits in a cell or cells that have not been contacted by the proteasome inhibitor.

Illustrative examples of proteasome inhibitors that can be used in particular embodiments contemplated herein include, but are not limited to, bortezomib (Velcade, PS-341), carfilzomib (Kyprolis), Oprozomib (ONX 0912), delanzomib (CEP-18770), ixazomib citrate (MLN9708), marizomib (NPI-0052; salinosporamide A), dihydroeponemycin, epoxomicin, ONX-914 (PR-957), syringolin A, TMC-95A, argryin A, disulfiram, epigallocatechin-3-gallate, MG-132, lactacystin, HBX41108, MG-262, MG-115, AM114, MLN2238, AM114, gliotoxin, P005091, PSI, omuralide, AdaAhx3L3VS, 8-hydroxyquinoline hemisulfate salt hemihydrate, and clasto-lactacystin β-lactone.

In particular embodiments, the proteasome inhibitor is carfilzomib.

### E. Glucocorticoids

Glucocorticoids contemplated herein are useful for treating cancer in a subject diagnosed with hematological malignancies. In particular embodiments, glucocorticoid treatment of hematological malignancies is combined with treatment with proteasome inhibitors, arsenic-containing compounds, and ascorbic acid or derivatives thereof. Without wishing to be bound to any particular theory, glucocorticoids may exert anticancer effects, in part, through activation of endogenous steroid receptors, including but not limited to glucocorticoid receptors.

In particular embodiments, a glucocorticoid activates a steroid receptor, e.g., glucocorticoid receptor to increase steroid receptor activity about 5%, about 10%, about 15%, about 20%, about 25%, about 30%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, or about two-fold, about three-fold, four-fold, about five-fold, about ten-fold, about twenty-fold, about thirty-fold, about forty-fold, about fifty-fold, about sixty-fold, about seventy-fold, about eighty-fold, about ninety-fold, or about one hundred-fold or greater. Activity of the steroid receptor may be determined by one or more methods including, but not limited to, measuring translocation of the steroid receptor from one cellular compartment to another (e.g. translocation from cytosol to the nucleus), measuring the gene product of a gene that is transcribed as a result of steroid receptor activation, or measuring amount of steroid receptor in an active confirmation, as compared to steroid receptors that are not contacted by the agent. Illustrative examples of glucocorticoids and glucocorticoid receptor agonists suitable for use in the compositions and methods contemplated herein include, but are not limited to, medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol, as well as combinations thereof.

In certain embodiments, the glucocorticoid administered to a subject is dexamethasone.

### F. Arsenic-Containing Compounds

While the toxicity of arsenic has long been known, arsenic has been used in traditional medicines for more than 2,000 years. In the 18^{th} century, a solution of arsenic trioxide and potassium bicarbonate, known as Fowler's solution, was created to treat infectious and malignant disease. Its efficacy in suppressing white cells was first described in 1878. Arsenic trioxide was therefore used to treat chronic myelogenous leukemia until more potent cytotoxic drugs superseded it in the 1940s. However, there was a resurgence of interest in such therapy, when arsenic trioxide was found to induce apoptosis and differentiation in acute promyelocytic leukemia cells in vitro. Similar benefits were later observed in in vitro experiments with multiple myeloma cells as well. Arsenic trioxide is currently approved by the FDA for treatment for acute promyelocytic leukemia, and clinical trials are currently underway to examine the effectiveness of arsenic trioxide treatment for other cancers.

There is currently no clear molecular mechanism of how arsenic-containing compounds are beneficial for cancer treatment. However, in vitro studies have shown that arsenic trioxide can induce apoptosis, alter the cellular redox state, disrupt mitochondrial function, and trigger activation of caspase proteins in cancer cells. Further, arsenic trioxide can slow or halt cell division in cancer cells by increasing levels of proteins that trigger cell cycle arrest. Without being bound to any particular theory, the present invention contemplates, in part, that arsenic-containing compounds combined with proteasome inhibitors, glucocorticoids, and ascorbic acid or derivatives thereof, synergistically treat a hematological malignancy.

As used herein, “arsenic-containing compound” refers to, in a pharmaceutically acceptable form, a compound which comprises arsenic. The present invention encompasses all pharmaceutically active species of arsenic, regardless of whether such species are organic or inorganic molecules, salts or solutions thereof, or mixtures thereof. The term “arsenic-containing compound” also encompasses hydrated versions, such as aqueous solutions, hydrolyzed products or ionized products of these compounds; and these compounds may contain different numbers of attached water molecules. In particular embodiments, any arsenic-containing compound(s), when used alone or in combination with other compounds, that can alleviate, reduce, ameliorate, prevent, or place or maintain in a state of remission of clinical symptoms or diagnostic markers associated with hematological malignancies can be used in the present invention.

Illustrative examples of arsenic-containing compounds include, but are not limited to, arsenic trioxide (As₂O₃), arsenic pentoxide (As₂O₅), arsenic hexoxide As₄O₆), arsenic triselenide (As₂Se₃), arsenic disulfide (As₂S₂), arsenic trisulfide (As₂S₃), arsenic pentasulfide (As₂O₅), arsenic tritelluride (As₂Te₃), sodium arsenate (Na₂HAsO₄), potassium arsenate (KH₂AsO₄), and sodium arsenyl tartrate (NaC₄H₄AsO₆).

In particular embodiments, the arsenic-containing compound arsenic trioxide.

### G. Ascorbic Acid and its Derivatives

Ascorbic acid, also known as vitamin C, is a strong water-soluble antioxidant. In the 1950's, ascorbic acid was proposed as treatment for cancer. Early proponents believed that treatment with ascorbic acid could slow the spread of tumors by stimulating collagen production or by inhibiting extracellular enzymes. While such mechanisms were not proven, initial studies that administered ascorbic acid orally and intravenously provided some benefit to cancer patients. However, carefully controlled double bind studies form the Mayo clinic in the late 1970's and early 1980's investigating oral administration of ascorbic acid failed to detect any such benefit, and interest in ascorbic acid as an anticancer treatment waned. Later work found that the maximum serum levels achieved by oral administration are generally lower than the levels achieved through intravenous administration, so intravenous ascorbic acid administration was tested with cancer treatments. Results from clinical trials suggested that intravenous ascorbic acid may benefit cancer patients. However, the contribution of ascorbic acid in these therapies was not determined, and the mechanism of how ascorbic acid affects cancer cells is not yet characterized.

Ascorbic acid has been shown to enhance the anti-cancer effects of arsenic-containing compounds. Without wishing to be bound to any particular theory, this effect is thought to be mediated through the oxidation of intracellular glutathione and results in accumulation of more reactive oxygen species which can increase arsenic-containing compounds' anti-cancer effects.

In particular embodiments of the present invention, treatment of hematological malignancies with ascorbic acid or derivatives thereof is combined with a proteasome inhibitor, a glucocorticoid, and an arsenic-containing compound.

As used herein, the term “ascorbic acid” includes the anionic component, ascorbate, whether as an acid or one of the pharmaceutically acceptable salts thereof, including sodium ascorbate and calcium ascorbate, any of which are included in a reference to “ascorbic acid.”

Derivatives of ascorbic acid include, but are not limited to, modifications to the molecule for the purposes of increasing lipophilicity, stability, or potency. The term “ascorbic acid derivative” or ascorbic acid and “derivatives thereof,” refer to structural variants of ascorbic acid that have a similar biological activity. Ascorbic acid derivatives have been cited as having ascorbic acid activity include compounds listed in (Warnat, et al., U.S. Pat. No. 2,150,140, Weisblatt, U.S. Pat. No. 2,454,747, Edwin, et al., U.S. Pat. No. 2,454,749, Kobayashi, et al., U.S. Pat. No. 3,318,914, Hoseney, et al., U.S. Pat. No. 4,044,154 and Markham, WIPO Application No. WO 1987001702, WIPO Application No. WO 1982000644, and Ivanov et al., WIPO application No. WO 2003017942), each of which is incorporated herein by reference in its entirety.

Illustrative examples of ascorbic acid derivatives suitable for use in particular embodiments contemplated herein include, but are not limited to, L-ascorbic acid-2-pyrophosphate esters, L-ascorbic acid-2-triphosphate esters, L-ascorbic acid-2-polyphosphate esters, sodium L-ascorbic acid-2-phosphate-6-palmitate, L-ascorbic acid-2-phosphate-6-palmitate, L-ascorbic acid-2-phosphate-6-stearate, L-ascorbic acid-2-phosphate-6-oleate and L-ascorbic acid-2-phosphate-6-arachidonate, 5,6-0-isoalkylidene ascorbic acid, 5,6-0-isopropylidine ascorbic acid, 5,6-O-benzylidene-L-ascorbic acid, 2-chloroethyl isopropylidene ascorbic acid, and L-ascorbate 2-sulphate.

In one embodiment, the subject is administered ascorbic acid.

### H. Pharmaceutical Compositions and Formulations

Compositions (i.e., medicaments) contemplated herein include, but are not limited to pharmaceutical compositions. A “pharmaceutical composition” refers to a formulation of a composition with one or more pharmaceutically acceptable carriers, diluents or excipients generally accepted in the art for the delivery of a compound or drug to a mammal, e.g., humans. In particular embodiments, pharmaceutical compositions comprise a proteasome inhibitor, a glucocorticoid, an arsenic containing compound, and/or ascorbic acid or a derivative thereof, formulated with one or more pharmaceutically-acceptable carriers, diluents, and/or excipients. It will also be understood that, if desired, the compositions of the invention may be administered in combination with other agents as well, such as, e.g., nucleic acids, proteins, small molecules, or pharmaceutically-active agents, adjunct therapies, etc. so long as the desired therapeutic effect is achieved. There is virtually no limit to other reagents that may also be included in the compositions, provided that the additional reagents do not adversely affect the desired cancer therapy.

In particular embodiments, compositions comprise pharmaceutically acceptable formulations with therapeutically effective amounts of proteasome inhibitors, glucocorticoids, arsenic-containing compounds, and/or ascorbic acid or derivatives thereof; or prodrugs, solvates, stereoisomers, racemates, or tautomers of proteasome inhibitors, glucocorticoids, arsenic-containing compounds, and/or ascorbic acid or derivatives thereof, formulated with one or more pharmaceutically acceptable carriers (additives), other active agents, and/or diluents.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin;

talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compounds contemplated herein exist in free base or acid form and can be converted to their pharmaceutically acceptable salts by treatment with the appropriate inorganic or organic base or acid by methods known to one skilled in the art. “Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

Salts of the compounds of the invention can be converted to their free base or acid form by standard techniques.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In particular embodiments, a pharmaceutical composition contemplated herein is formulated so as to allow the active ingredients contained therein to be bioavailable upon administration of the composition to a subject. In one embodiment, pharmaceutical compositions can be prepared by combining proteasome inhibitors, glucocorticoids, arsenic-containing compounds, and/or ascorbic acid or derivatives thereof, with an appropriate pharmaceutically acceptable carrier, diluent or excipient, and may be formulated into preparations in solid, semi solid, liquid, gels, and microspheres. However, in certain embodiments the subject compounds may be simply dissolved or suspended in sterile water of physiological saline, Ringer's solution, or 0.9% NaCl.

Solid formulations of the compositions contemplated herein, include dragees, capsules, pills and granules, optionally scored or prepared with coatings and shells, such as enteric coatings and other coatings. Solid dosage forms may also be formulated so as to provide slow or controlled release of the compound. Thus, solid formulations could include any material that could provide a desired release profile of the compound, including but not limited to hydroxypropylmethyl cellulose in varying proportions, or other polymer matrices, liposomes and/or microspheres.

Coated, gel, or encapsulating formulations of proteasome inhibitors, glucocorticoids, arsenic-containing compounds, and/or ascorbic acid or derivatives thereof may also be formulated to deliver pulsatile, sustained, or extended release. For example, one method of pulsatile release could be achieved by layering multiple coatings of proteasome inhibitors, glucocorticoids, arsenic-containing compounds, and/or ascorbic acid or derivatives thereof, or by incorporating proteasome inhibitors, glucocorticoids, arsenic containing compounds, and/or ascorbic acid or derivatives thereof within different regions of the formulation having different release times.

Liquid dosage formulations contemplated herein include pharmaceutically acceptable emulsions, microemulsions, solutions, suspensions, syrups and elixirs. In addition, the liquid dosage formulations may contain inert diluents commonly used in the art, including but not limited to water or other solvents; solubilizing agents and emulsifiers, such as ethyl alcohol, isopropyl alcohol, ethyl carbonate, ethyl acetate, benzyl alcohol, benzyl benzoate, propylene glycol, 1,3-butylene glycol; oils (in particular, cottonseed, groundnut, corn, germ, olive, castor and sesame oils); glycerol; tetrahydrofuryl alcohol; polyethylene glycols; and fatty acid esters of sorbitan, and mixtures thereof.

Suspensions formulations include, without limitation, ethoxylated isostearyl alcohols; polyoxyethylene sorbitol and sorbitan esters; microcrystalline cellulose; aluminum metahydroxide; bentonite; agar-agar; tragacanth; and mixtures thereof.

Injectable depot formulations can be made by forming microencapsulated matrices of the composition in biodegradable polymers. Examples of biodegradable polymers include, but are not limited to polylactide-polyglycolide, poly(orthoesters) and poly(anhydrides). The ratio of composition to polymer and the nature of the particular polymer employed can affect the rate of release of proteasome inhibitors, glucocorticoids, arsenic-containing compounds, and/or ascorbic acid or derivatives thereof from the composition. Depot injectable formulations can also be prepared by entrapping the drug in liposomes or microemulsions.

Proper fluidity of liquid, suspension and other formulations of the compounds can be maintained by the use of coating materials such as lecithin; by the maintenance of the required particle size in the case of dispersions; or by the use of surfactants.

Formulations may also include anti-contamination agents for the prevention of microorganism contamination. Anti-contamination agents may include but are not limited to antibacterial and antifungal agents, for example, paraben, chlorobutanol, phenol sorbic acid, antibiotics, and the like.

Formulations may also be sterilized by, for example, by filtration through a bacteria-retaining filter, or by incorporating sterilizing agents in the form of sterile solid formulations which can be dissolved in sterile water, or some other sterile medium immediately before use or formulation.

Formulations may also be endotoxin free. As used herein, the term “endotoxin free” refers to compositions or formulations that contain at most trace amounts (i.e., amounts having no adverse physiological effects to a subject) of endotoxin, and preferably undetectable amounts of endotoxin. By “substantially free of endotoxin” is meant that there is less endotoxin per dose of cells than is allowed by the FDA for a biologic, which is a total endotoxin of 5 EU/kg body weight per day, which for an average 70 kg person is 350 EU per total dose of cells. In one embodiment, the term “endotoxin free” refers to a composition or formulation that is at least 95%, at least 96%, at least 97%, at least 98%, at least 99%, or 100% endotoxin free. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipooligosaccharides (LOS) found in the outer membrane of various Gram-negative bacteria, and which represent a central pathogenic feature in the ability of these bacteria to cause disease. Small amounts of endotoxin in humans can produce fever, a lowering of the blood pressure, and activation of inflammation and coagulation, among other adverse physiological effects. Therefore, it is often desirable to remove most or all traces of endotoxin from drug product containers, because even small amounts may cause adverse effects in humans.

Pharmaceutical compositions may further comprise one or more components that enhance the bioavailability of the active ingredients of the composition, e.g., penetration enhancers, stabilizing agents, and one or more components that provide slow or controlled release of the proteasome inhibitors, glucocorticoids, arsenic-containing compounds, and/or ascorbic acid or derivatives thereof in a composition, e.g., biocompatible polymers and/or gels.

In particular embodiments, compositions comprising penetration enhancers will facilitate the delivery of the composition across biological barriers. A “penetration enhancer” or “permeability enhancer” includes a polyol such as polyethylene glycol (PEG), glycerol (glycerin), maltitol, sorbitol etc.; diethylene glycol monoethyl ether, azone, benzalkonium chloride (ADBAC), cetylperidium chloride, cetylmethylammonium bromide, dextran sulfate, lauric acid, menthol, methoxysalicylate, oleic acid, phosphatidylcholine, polyoxyethylene, polysorbate 80, sodium glycholate, sodium lauryl sulfate, sodium salicylate, sodium taurocholate, sodium taurodeoxycholate, sulfoxides, sodium deoxycholate, sodium glycodeoxycholate, sodium taurocholate and surfactants such as sodium lauryl sulfate, laureth-9, cetylpyridinium chloride and polyoxyethylene monoalkyl ethers, benzoic acids, such as sodium salicylate and methoxy salicylate, fatty acids, such as lauric acid, oleic acid, undecanoic acid and methyl oleate, fatty alcohols, such as octanol and nonanol, laurocapram, cyclodextrins, thymol, limonene, urea, chitosan and other natural and synthetic polymers.

Suitable polyols for inclusion in the solutions include glycerol and sugar alcohols such as sorbitol, mannitol or xylitol, polyethylene glycol and derivatives thereof. In some embodiments the composition further includes a preservative. Accepted preservatives such as benzalkonium chloride and disodium edetate (EDTA) are included in the compositions of the invention in concentrations sufficient for effective antimicrobial action, about 0.0001 to 0.1%, based on the weight of the composition.

In particular embodiments, compositions comprise stabilizers to increase the therapeutic lifetime of the compositions in vivo. Exemplary stabilizers include fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In further embodiments, the chosen stabilizer changes the hydrophobicity of the formulation (e.g., oleic acid, waxes), or improves the mixing of various components in the formulation (e.g., ethanol), affects the moisture level in the formula (e.g., PVP or polyvinyl pyrrolidone), affects the mobility of the phase (substances with melting points higher than room temperature such as long chain fatty acids, alcohols, esters, ethers, amides etc. or mixtures thereof; waxes), and/or improves the compatibility of the formula with encapsulating materials (e.g., oleic acid or wax). In other embodiments, stabilizers are present in sufficient amounts to inhibit the degradation of the proteasome inhibitors, glucocorticoids, arsenic containing compounds, and/or ascorbic acid or derivatives thereof in a composition. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In particular embodiments, compositions are formulated as controlled release formulations. In general, controlled release drug formulations impart control over the release of drug with respect to site of release and time of release in vivo. Controlled release includes to immediate release, delayed release, sustained release, extended release, variable release, pulsatile release and bi-modal release. Advantages offered by controlled release include: less frequent dosing; more efficient drug utilization; localized drug delivery by placement of a delivery device or formulation at a treatment site in vivo; and the opportunity to administer and release two or more different drugs, each having a unique release profile, or to release the same drug at different rates or for different durations, by means of a single dosage unit.

Controlled release formulations may be made by formulating the compositions with biocompatible polymers, viscosity agents, gels, paints, foams, xerogels, microparticles, hydrogels, nanocapsules, and thermoreversible gels, or combinations thereof. In particular embodiments, the polymer or gels are biodegradable. Release properties are often controlled by the particular combination of polymers or gels used to formulate the composition. These methods are well known in the art.

Exemplary polymers suitable for formulating the inventive compositions include, but are not limited to polyamides, polycarbonates, polyalkylenes (polyethylene glycol (PEG)), polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In particular embodiments, the polymer is a ABA-type or BAB-type triblock copolymers or mixtures thereof, wherein the A-blocks are relatively hydrophobic and comprise biodegradable polyesters or poly(orthoester), and the B-blocks are relatively hydrophilic and comprise polyethylene glycol (PEG). The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof.

Exemplary viscosity agents suitable for use in formulating compositions include, but are not limited to, hydroxypropyl methylcellulose, hydroxyethyl cellulose, polyvinylpyrrolidone, carboxymethyl cellulose, polyvinyl alcohol, sodium chondroitin sulfate, sodium hyaluronate, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, or polyvinylpyrrolidone (PVP: povidone).

Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, glycerin-based gels, glycerin-derived compounds, conjugated, or crosslinked gels, matrices, hydrogels, and polymers, as well as gelatins and their derivatives, and various native and synthetic hydrogel and hydrogel-derived compounds, and any combinations or mixtures thereof.

In a particular embodiment, compositions contemplated herein comprise an effective amount of one or more of, or all of the following: proteasome inhibitors, glucocorticoids, arsenic containing compounds, and/or ascorbic acid or derivatives thereof, alone or in combination with one or more other therapeutic agents or modalities. Thus, the compositions may be administered individually or in combination with each other and/or with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, NSAIDs, DMARDs, anti-inflammatories, chemotherapeutics, radiotherapeutics, therapeutic antibodies, or other active and ancillary agents.

In certain embodiments, compositions contemplated herein may be administered in conjunction with any number of chemotherapeutic agents. Illustrative examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin and its pegylated formulations, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine;

pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on cancers such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the compositions contemplated herein. In one embodiment, the compositions contemplated herein are administered with nonsteroidal anti-inflammatory drugs (NSAIDS) including aspirin, ibuprofen, naproxen, methotrexate, sulfasalazine, leflunomide, anti-TNF medications, cyclophosphamide, and mycophenolate.

Other exemplary NSAIDs are chosen from the group consisting of ibuprofen, naproxen, naproxen sodium, COX-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers, cytokine inhibitors, such as the TNF antagonists, adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline. Illustrative examples of therapeutic antibodies suitable for combination with compositions contemplated herein, include but are not limited to, bavituximab, bevacizumab (avastin), bivatuzumab, blinatumomab, conatumumab, daratumumab, duligotumab, dacetuzumab, dalotuzumab, elotuzumab (HuLuc63), gemtuzumab, ibritumomab, indatuximab, inotuzumab, lorvotuzumab, lucatumumab, milatuzumab, moxetumomab, ocaratuzumab, ofatumumab, rituximab, siltuximab, teprotumumab, and ublituximab.

In certain embodiments, the compositions described herein are administered in conjunction with one or more cytokines. A “cytokine” refers to proteins released by one cell population that act on another cell as intercellular mediators. Illustrative examples of such cytokines are lymphokines, monokines, and traditional polypeptide hormones. Included among the cytokines are growth hormones such as human growth hormone, hepatic growth factor; tumor necrosis factor-alpha and -beta; mullerian-inhibiting substance; inhibin; activin; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In particular embodiments, the compositions contemplated herein comprise a concentration of one or more pharmaceutically active ingredients (i.e., proteasome inhibitors, glucocorticoids, arsenic-containing compounds, and/or ascorbic acid or derivatives thereof; and optionally pharmaceutically acceptable salts, prodrugs, solvates, stereoisomers, racemates, or tautomers thereof) of between about 0.01% to about 90%, between about 0.01% to about 50%, between about 0.1% to about 70%, between about 0.1% to about 50%, between about 0.1% to about 40%, between about 0.1% to about 30%, between about 0.1% to about 20%, between about 0.1% to about 10%, or between about 0.1% to about 5%, of each active ingredient, by weight of the composition.

In certain embodiments, the compositions described herein have a concentration of each active pharmaceutical agent between about 1% to about 50%, between about 5% to about 50%, between about 10% to about 40%, or between about 10% to about 30%, of the active ingredient, or pharmaceutically acceptable salt, prodrug, solvate, stereoisomer, racemate, or tautomer thereof, by weight of the composition.

In some embodiments, the formulations have a concentration of active pharmaceutical ingredient of between about 0.1 to about 70 mg/mL, between about 0.5 mg/mL to about 70 mg/mL, between about 0.5 mg/mL to about 50 mg/mL, between about 0.5 mg/mL to about 20 mg/mL, between about 1 mg to about 70 mg/mL, between about 1 mg to about 50 mg/mL, between about 1 mg/mL and about 20 mg/mL, between about 1 mg/mL to about 10 mg/mL, or between about 1 mg/mL to about 5 mg/mL, of the active agent, or pharmaceutically acceptable salt, prodrug, solvate, stereoisomer, racemate, or tautomer thereof, by volume of the formulation.

In one embodiment, the formulations additionally provide an immediate release of one or more pharmaceutically active ingredients (i.e., proteasome inhibitors, glucocorticoids, arsenic-containing compounds, and/or ascorbic acid or derivatives thereof, or pharmaceutically acceptable salts, prodrugs, solvates, stereoisomers, racemates, or tautomers thereof) from the composition, or within 1 minute, or within 5 minutes, or within 10 minutes, or within 15 minutes, or within 30 minutes, or within 60 minutes or within 90 minutes.

In another embodiment, a therapeutically effective amount of at least one pharmaceutically active ingredient is released from the composition immediately, or within 1 minute, or within 5 minutes, or within 10 minutes, or within 15 minutes, or within 30 minutes, or within 60 minutes or within 90 minutes.

In yet another embodiment, a composition is formulated as an extended release formulation. In certain embodiments, diffusion of at least one pharmaceutically active ingredient from the formulation occurs for a time period exceeding 5 minutes, 15 minutes, 30 minutes, 1 hour, 4 hours, 6 hours, 12 hours, 18 hours, 1 day, 2 days, 3 days, 4 days, 5 days, 6 days, 7 days, 10 days, 12 days, 14 days, 18 days, 21 days, 25 days, 30 days, 45 days, 2 months 3 months 4 months 5 months 6 months 9 months or 1 year.

In particular embodiments, a therapeutically effective amount of at least one pharmaceutically active ingredient is released from the formulation for a time period exceeding 5 minutes, 15 minutes, 30 minutes, 1 hour, 4 hours, 6 hours, 12 hours, 18 hours, 1 day, 2 days, 3 days, 4 days, 5 days, 6 days, 7 days, 10 days, 12 days, 14 days, 18 days, 21 days, 25 days, 30 days, 45 days, 2 months 3 months 4 months 5 months 6 months 9 months or 1 year.

In further embodiments, the formulation provides both an immediate release and an extended release formulation. In particular embodiments, the formulation contains a 0.25:1 ratio, a 0.5:1 ratio, a 1:1 ratio, a 1:2 ratio, a 1:3, a 1:4 ratio, a 1:5 ratio, a 1:7 ratio, a 1:10 ratio, a 1:15 ratio, or a 1:20 ratio of immediate release and extended release formulations. In a further embodiment the formulation provides an immediate release of a first pharmaceutically active ingredient and an extended release of a second pharmaceutically active ingredient or other therapeutic agent.

In additional embodiments, the formulation provides a 0.25:1 ratio, a 0.5:1 ratio, a 1:1 ratio, a 1:2 ratio, a 1:3, a 1:4 ratio, a 1:5 ratio, a 1:7 ratio, a 1:10 ratio, a 1:15 ratio, or a 1:20 ratio of immediate release and extended release formulations of one or more pharmaceutically active ingredients.

The combination of immediate release, delayed release and/or extended release compositions or formulations may be combined with other pharmaceutical agents, as well as the excipients, diluents, stabilizers, carrier agents and other components disclosed elsewhere herein. As such, depending upon the components of a composition, the thickness or viscosity desired, or the mode of delivery chosen, alternative aspects of the embodiments disclosed herein are combined with the immediate release, delayed release and/or extended release embodiments accordingly.

Additional methods of formulating compositions are known to the skilled artisan, for example, as described in the Physicians Desk Reference, 62nd edition. Oradell, N J: Medical Economics Co., 2008; Goodman & Gilman's The Pharmacological Basis of Therapeutics, Eleventh Edition. McGraw-Hill, 2005; Remington: The Science and Practice of Pharmacy, 20th Edition. Baltimore, Md.: Lippincott Williams & Wilkins, 2000; and The Merck Index, Fourteenth Edition. Whitehouse Station, N.J.: Merck Research Laboratories, 2006; each of which is hereby incorporated by reference in relevant parts.

### I. Administration

In particular embodiments, a method of treating a subject with a hematological malignancy is contemplated comprising administering to the subject a proteasome inhibitor, a glucocorticoid, an arsenic-containing compound, and ascorbic acid or a derivative thereof. Compositions contemplated herein may be administered as one or more solids, semi-solids, gels, or liquids, or combination thereof. For example, proteasome inhibitors, glucocorticoids, arsenic-containing compounds, and/or ascorbic acid or derivatives thereof and other pharmaceutically active agents may be individually formulated for intravenous administration in a liquid dosage form or for oral administration as a single tablet or capsule or as a combination of one or more tablets, capsules, or other dosage forms. The specific amount/dosage regimen will vary depending on the weight, gender, age and health of the individual; the formulation, the biochemical nature, bioactivity, bioavailability and the side effects of the agents and the number and identity of the agents in the complete therapeutic regimen.

As used herein, the terms “administering,” “administer,” or “administration” refer to deliver one or more compounds or compositions to a subject parenterally, enterally, or topically. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration.

In particular embodiments, an agent or composition is administered parenterally, optionally by intravenous administration or oral administration to a subject.

In various embodiments, the development of suitable dosing and treatment regimens for using the particular compositions contemplated herein in a variety of treatment regimens including, e.g., oral, parenteral, intravenous, intranasal, and intramuscular administration and formulation, is well known in the art.

In particular embodiments, proteasome inhibitors, glucocorticoids, arsenic-containing compounds, and/or ascorbic acid or derivatives thereof are administered orally to a subject. The agent can be administered to the subject at a dose in the range of about 1-100 mg, about 1-50 mg, about 50-100 mg, about 1-5 mg, about 5-10 mg, about 10-15mg, about 15-20 mg, about 20-30 mg, about 30-40 mg, about 40-50 mg, about 50-60 mg, about 60-70 mg, about 70-80 mg, about 80-90 mg, or about 90-100 mg or more. In certain embodiments, the agent is administered in a dose of about 1 mg, about 2 mg, about 2.5 mg, about 3 mg, about 4 mg, about 5 mg, about 6 mg, about 7 mg, about 8 mg, about 9 mg, about 10 mg, about 15 mg, about 20 mg, about 25 mg, about 50 mg, or about 100 mg or more. In some embodiments of the invention, an oral dose of an agent is administered to the subject at least once in a treatment cycle, at least once in a 28 day treatment cycle, at least once a week, at least once every other day, at least once a day, or at least twice a day.

In particular embodiments, a proteasome inhibitor, a glucocorticoid, an arsenic-containing compounds, and/or ascorbic acid or a derivative thereof is administered intravenously. The agent can be administered intravenously at a dose of about 0-100 mg, about 1 -50 mg, about 50-100 mg, about 1-10 mg, about 10-20 mg, about 20-30 mg, about 30-40 mg, about 40-50 mg, about 50-60 mg, about 60-70 mg, about 70-80 mg, about 80-90 mg, or about 90-100 mg or more. In certain embodiments, the intravenous dose of agent is about one mg, about two mg, about three mg, about four mg, about five mg, about six mg, about seven mg, about eight mg, about nine mg, about ten mg, about 15 mg, about 20 mg, about 25 mg, about 30 mg, about 35 mg, about 40 mg, about 45 mg, about 50 mg, about 60 mg, about 70 mg, about 80 mg, about 90 mg, or about 100 mg or more. Doses of agents can be delivered intravenously in any pharmaceutically suitable vehicles for injection or infusion known in the art.

In some embodiments, the agent can be administered intravenously at a dose of about 0-100 mg/m², about 1-50 mg/m², about 50-100 mg/m², about 1-10 mg/m², about 10-20 mg/m², about 20-30 mg/m², about 30-40 mg/m², about 40-50 mg/m², about 50-60 mg/m², about 60-70 mg/m², about 70-80 mg/m², about 80-90 mg/m², or about 90-100 mg/m² or more. In certain embodiments, the intravenous dose of agent is about one mg/m², about two mg/m², about three mg/m², about four mg/m², about five mg/m², about six mg/m², about seven mg/m², about eight mg/m², about nine mg/m², about ten mg/m², about 15 mg/m², about 20 mg/m², about 25 mg/m², about 30 mg/m², about 35 mg/m², about 40 mg/m², about 45 mg/m², about 50 mg/m², about 60 mg/m², about 70 mg/m², about 80 mg/m², about 90 mg/m², or about 100 mg/m² or more.

In some embodiments, the agent can be administered intravenously at a dose of about 0-10 mg/kg, about 0-5 mg/kg, about 5-10 mg/kg, about 0-1 mg/kg, about 1-2 mg/kg, about 2-3 mg/kg, about 3-4 mg/kg, about 4-5 mg/kg, about 5-6 mg/kg, about 6-7 mg/kg, about 7-8 mg/kg, about 8-9 mg/kg, or about 9-10 mg/kg or more. In certain embodiments, the intravenous dose of agent is about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about one mg/kg, about two mg/kg, about three mg/kg, about four mg/kg, about five mg/kg, about six mg/kg, about seven mg/kg, about eight mg/kg, about nine mg/kg, or about ten mg/kg or more.

In some embodiments, a proteasome inhibitor, a glucocorticoid, an arsenic-containing compound, and ascorbic acid or a derivative thereof are each administered at least once during a treatment cycle. In some embodiments, a proteasome inhibitor, a glucocorticoid, an arsenic-containing compound, and ascorbic acid or a derivative thereof are administered to the subject on the same days. In some embodiments, a proteasome inhibitor, a glucocorticoid, an arsenic containing compound, and ascorbic acid or a derivative thereof are administered to the subject on the different days. In some embodiments, a proteasome inhibitor, a glucocorticoid, an arsenic-containing compound, and ascorbic acid or a derivative thereof are administered to the subject on the same days and on different days according to treatment schedules.

In particular embodiments, an agent is administered to the subject over one or more treatment cycles. A treatment cycle can be at least two, at least three, at least four, at least five, at least six, at least seven, at least 14, at least 21, at least 28, at least 48, or at least 96 days or more. In one embodiment, a treatment cycle is 28 days. In certain embodiments, the agents are administered over the same treatment cycle or concurrently over different treatment cycles assigned for each agent. In various embodiments, the treatment cycle is determined by a health care professional based on conditions and needs of the subject.

In some embodiments, an agent is administered on at least one day, at least two days, at least three days, at least four days, at least five days, at least six days, at least seven days, at least eight days, at least nine days, at least ten days, at least eleven days, at least twelve days, at least 13 days, at least 14 days, at least 21 days, or all 28 days of a 28 day treatment cycle. In particular embodiments, an agent is administered to a subject once a day. In other particular embodiments, an agent is administered twice a day. In certain embodiments an agent is administered more than twice a day.

In particular embodiments, an agent is administered on day 1, day 2, day 8, day 9, day 15, and day 16 of a 28 day treatment cycle. In some embodiments, a proteasome inhibitor, a glucocorticoid, an arsenic-containing compound, and ascorbic acid or a derivative thereof is administered on day 1, day 2, day 8, day 9, day 15, and day 16 of a 28 day treatment cycle. In some embodiments, a glucocorticoid is administered after a proteasome inhibitor is administered on day 1, day 2, day 8, day 9, day 15, and day 16 of a 28 day treatment cycle. In some embodiments, ascorbic acid or a derivative thereof is administered after an arsenic-containing compound is administered on administered on day 1, day 2, day 8, day 9, day 15, and day 16 of a 28 day treatment cycle.

The number of times a composition is administered to a subject in need thereof depends on the discretion of a medical professional, the disorder, the severity of the disorder, and the subject's response to the formulation. In some embodiments, a composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the composition may administered continuously; or, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

### J. Methods

In various embodiments, methods of treating, preventing, or ameliorating at least one symptom of a hematological malignancy are provided. Illustrative examples of hematological malignancies suitable for treatment with the compositions and methods contemplated herein include, but are not limited to multiple myeloma, leukemia, or lymphoma. Leukemias can include, but are not limited to, acute lymphoblastic leukemia, acute myelogenous leukemia, chronic lymphocytic leukemia, chronic myelogenous leukemia, acute monocytic leukemia. Lymphomas can include, but are not limited to, Hodgkin's lymphomas, such as nodular sclerosis Hodgkin's lymphoma, mixed cellularity subtype Hodgkin's lymphoma, Lymphocyte rich Hodgkin's lymphoma, and lymphocyte depleted Hodgkin's Lymphoma; and non-Hodgkin's lymphoma, such as diffuse large B-cell lymphoma, primary mediastinal B-cell lymphoma, follicular lymphoma, chronic lymphocytic leukemia, mantle cell lymphoma, marginal zone B-cell lymphomas, Burkitt lymphoma, lymphoplasmacytic lymphoma, primary central nervous system lymphoma, and T-cell lymphomas.

Myelomas include, but are not limited to, multiple myeloma. Without wishing to be bound to any particular theory, it is contemplated that the treatment of a hematological malignancy with the compositions contemplated herein offer patients with relapsed or refractory cancers an improved therapeutic outcome and increased chance of long-term survival.

In various embodiments, the methods contemplated herein comprise administration of a proteasome inhibitor and one or more other pharmaceutically acceptable agents to synergistically treat a hematological malignancy. In one embodiment, a proteasome inhibitor is combined with a glucocorticoid, an arsenic-containing compound, and ascorbic acid or a derivative thereof to synergistically treat a hematological malignancy.

In particular embodiments, methods of preventing, ameliorating one or more symptoms of, or treating a subject having multiple myeloma are provided. In particular embodiments, the methods comprise administering a proteasome inhibitor and one or more other pharmaceutically acceptable agents. In certain embodiments, a subject having multiple myeloma is administered a proteasome inhibitor with a glucocorticoid, an arsenic-containing compound, and ascorbic acid or a derivative thereof.

In certain embodiments, methods of preventing, ameliorating one or more symptoms of, or treating a subject having a relapsed hematological malignancy, e.g, multiple myeloma, are provided. In some embodiments, a method of treating a subject with a relapsed hematological malignancy comprises administering a proteasome inhibitor and one or more other pharmaceutically acceptable agents. In other embodiments, a subject having a relapsed hematological malignancy is administered a proteasome inhibitor with a glucocorticoid, an arsenic-containing compound, and ascorbic acid or a derivative thereof.

In some embodiments, methods of preventing, ameliorating one or more symptoms of, or treating a subject having a hematological malignancy that is refractory to one or more prior cancer treatments are provided. In some embodiments, a method of treating a subject with a refractory hematological malignancy comprises administering a proteasome inhibitor and one or more other pharmaceutically acceptable agents. In other embodiments, a subject having a refractory hematological malignancy is administered a proteasome inhibitor with and a glucocorticoid, an arsenic-containing compound, and ascorbic acid or a derivative thereof.

In particular embodiments, the relapsed or refractory malignancy was previously treated with one or more of a chemotherapeutic agent, e.g., doxorubicin; a proteasome inhibitor, e.g., bortezomib, carfilzomib; an arsenic containing compound, e.g. arsenic trioxide; ascorbic acid or a derivative thereof; thalidomide or a derivative thereof, e.g., pomalidomide, lenalidomide; a glucocorticoid, e.g., dexamethasone, methylprednisone, methylprednisolone; or other agents including but not limited to alkylating agents, anthracyclines, taxanes, histone deacetylase inhibitors, topoisomerase inhibitors, kinase inhibitors, monoclonal antibodies, nucleotide analogs and precursor analogs, peptide antibiotics, retinoids, and vinca alkaloids. In one embodiment, the relapsed or refractory malignancy was previously treated with a proteasome inhibitor and a glucocorticoid. In another embodiment, the relapsed or refractory malignancy was not previously treated with a proteasome inhibitor, a glucocorticoid, an arsenic-containing compound, and ascorbic acid or a derivative thereof.

In particular embodiments, a subject having a hematological malignancy is administered a proteasome inhibitor selected from the group consisting of: bortezomib (Velcade, PS-341), carfilzomib (Kyprolis), Oprozomib (ONX 0912), delanzomib (CEP-18770), ixazomib citrate (MLN9708), marizomib (NPI-0052; salinosporamide A), dihydroeponemycin, epoxomicin, ONX-914 (PR-957), syringolin A, TMC-95A, argryin A, disulfiram, epigallocatechin-3-gallate, MG-132, lactacystin, HBX41108, MG-262, MG-115, AM114, MLN2238, AM114, gliotoxin, P005091, PSI, omuralide, AdaAhx3L3VS, 8-hydroxyquinoline hemisulfate salt hemihydrate, and clasto-lactacystin β-lactone; a glucocorticoid; an arsenic containing compound, and ascorbic acid or a derivative thereof.

In certain embodiments, a subject having a hematological malignancy is administered a proteasome inhibitor; a glucocorticoid selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol; an arsenic containing compound and ascorbic acid or a derivative thereof.

In various embodiments, a subject having a hematological malignancy is administered a proteasome inhibitor; a glucocorticoid; an arsenic-containing compound selected from the group consisting of arsenic trioxide (As₂O₃), arsenic pentoxide (As₂O₅), arsenic hexoxide As₄O₆), arsenic triselenide (As₂Se₃), arsenic disulfide (As₂S₂), arsenic trisulfide (As₂S₃), arsenic pentasulfide (As₂O₅), arsenic tritelluride (As₂Te₃), sodium arsenate (Na₂HAsO₄), potassium arsenate (KH₂AsO₄), and sodium arsenyl tartrate (NaC₄H₄AsO₆); and ascorbic acid or a derivative thereof.

In various embodiments, a subject having a hematological malignancy is administered a proteasome inhibitor; a glucocorticoid; an arsenic-containing compound; and ascorbic acid or a derivative thereof selected from the group consisting of L-ascorbic acid-2-pyrophosphate esters, L-ascorbic acid-2-triphosphate esters, L-ascorbic acid-2-polyphosphate esters, sodium L-ascorbic acid-2-phosphate-6-palmitate, L-ascorbic acid-2-phosphate-6-palmitate, L-ascorbic acid-2-phosphate-6-stearate, L-ascorbic acid-2-phosphate-6-oleate and L-ascorbic acid-2-phosphate-6-arachidonate, 5,6-0-isoalkylidene ascorbic acid, 5,6-0-isopropylidine ascorbic acid, 5,6-O-benzylidene-L-ascorbic acid, 2-chloroethyl isopropylidene ascorbic acid, and L-ascorbate 2-sulphate.

In particular embodiments, a subject having a hematological malignancy is administered a proteasome inhibitor selected from the group consisting of bortezomib, carfilzomib, oprozomib, ixazomib citrate, marizomib, delanzomib; a glucocorticoid selected from the group consisting of dexamethasone, methylprednisone, and methylprednisolone; arsenic trioxide, and ascorbic acid. It is understood that any agent from each class can be combined with any other agent from a different class. In additional embodiments, a subject is also administered one or more additional pharmaceutically active agents.

In particular embodiments, a subject having a hematological malignancy is administered carfilzomib, dexamethasone, arsenic trioxide, and ascorbic acid. In a certain embodiment, a subject having a hematological malignancy is administered carfilzomib, dexamethasone, arsenic trioxide, and ascorbic acid intravenously. In one embodiment, carfilzomib, dexamethasone, arsenic trioxide, and ascorbic acid are administered to a subject on day 1, day 2, day 8, day 9, day 15, and day 16 or a 28 day treatment cycle.

All publications, patent applications, and issued patents cited in this specification are herein incorporated by reference as if each individual publication, patent application, or issued patent were specifically and individually indicated to be incorporated by reference in its entirety.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Combined Proteasome Inhibitor, Arsenic Trioxide, Glucocorticoid, and Intravenous Ascorbic Acid Therapy for Multiple Meyloma

The efficacy and safety of the combination of intravenous carfilzomib, intravenous arsenic trioxide, intravenous dexamethasone, and intravenous ascorbic acid (KADA therapy) for multiple myeloma were evaluated in patients who were refractory to previous treatments with combination therapies that included intravenous carfilzomib and glucocorticoids.

Nine patients with refractory multiple myeloma were treated; all had been previously treated with at least 1 carfilzomib-containing regimen and were refractory to the most recent carfilzomib-containing regimen. One patient had previously been treated with another intravenous arsenic trioxide-containing regimen without carfilzomib. Treatment consisted of carfilzomib 27 mg/m² intravenously on days 1, 2, 8, 9, 15, and 16, arsenic trioxide 0.25 mg/kg intravenously on days 1, 2, 8, 9, 15, and 16 of a 28-day cycle with ascorbic acid 1,000 mg intravenously after each arsenic trioxide treatment and dexamethasone 40mg (one patient only received 4 mg) intravenously on days 1, 8, 15, and 22.

The patient data is summarized in Table 1.

TABLE 1

| Patient | | | | Heavy | Light | | | Prior |
|---|---|---|---|---|---|---|---|---|
| ID | | | Diagnosis | Chain | Chain | | | Lines of |
| Number | Sex | Age | (DX) | Isotype | Isotype | Dx Date | ISS | Therapy |
| 1027 | M | 73 | MM | IgG | Kappa | Oct. 9, 2001 | II | 7 |
| 1057 | F | 66 | MM | N/P | Lambda | Oct. 11, 2011 | I | 5 |
| 1119 | M | 44 | MM | IgG | Lambda | Jan. 21, 2004 | I | 6 |
| 1525 | F | 58 | MM | N/P | Kappa* | Aug. 1, 2006 | Unknown | 9 |
| 1701 | M | 57 | MM | IgA | Kappa | Jul. 20, 2008 | III | 7 |
| 1702 | M | 57 | MM | IgG | Kappa | Jul. 25, 2008 | I | 10 |
| 1795 | F | 71 | MM | N/P | Kappa | Dec. 8, 2008 | II | 12 |
| 2233 | F | 54 | MM | N/P | Kappa | Nov. 9, 2011 | II | 7 |
| 2417 | M | 62 | MM | N/P | Kappa | Mar. 21, 2013 | II | 3 |

Patients ranged from 44 to 73 years of age (median: 58) and had received 3 to 12 prior lines of therapy (median: 7). Of the nine multiple myeloma patients, five (55.6%) were men and four (44.4%) were women. Patients ranged from 44 to 73 years of age (median: 58). Two patients had IgG kappa disease, one had IgG lambda disease, one had IgA kappa disease, four had kappa light chain disease and one had lambda light chain disease. Patients were diagnosed between Oct. 9, 2001 and Mar. 21, 2013. At the time of MM diagnosis, 3 patients were ISS stage I, 4 patients were ISS stage II, and 1 patient was ISS stages III. The staging of one patient could not be assessed. The number of prior lines of therapies received ranged from 3 to 12 (median: 7).

The following Table summarizes the treatment data for the patients during the study.

TABLE 2

| Patient | | Best Re- | | |
|---|---|---|---|---|
| ID | KADA | sponse (BR) | Duration of | KADA End |
| Number | Start Date | to KADA | BR in months | Date |
| 1027* | Sep. 11, 2013 | MR | 5+ | ongoing |
| 1057 | Dec. 17, 2013 | PD | N/A | Feb. 11, 2014 |
| 1119 | Nov. 25, 2013 | MR | 3 | Feb. 18, 2014 |
| 1525 | Aug. 27, 2013 | CR | 7+ | ongoing |
| 1701 | Dec. 16, 2013 | MR | 1 | Mar. 4, 2014 |
| 1702 | Sep. 24, 2013 | PD | N/A | Oct. 14, 2013 |
| 1795 | Sep. 12, 2013 | PR | 1 | Oct. 31, 2013 |
| 2233 | Nov. 19, 2013 | SD | 0.5 | Jan. 9, 2014 |
| 2417 | Dec. 12, 2013 | PD | N/A | Jan. 27, 2014 |

The overall clinical benefit rate was 55.6%, which included one (11.1%) complete response (CR), one (11.1%) partial response (PR) and three (33.3%) minimal responses (MR). One (11.1%) achieved stable disease (SD) and three (33.3%) showed progressive disease (PD) on the combined treatment. Two patients discontinued therapy due to tolerability issues after having achieved MR and SD. The one patient who had previously received arsenic trioxide achieved an MR on KADA.

The regimen was generally well-tolerated but two patients discontinued therapy due to tolerability issues. Both patients who discontinued therapy due to poor tolerability reported malaise and fatigue. One patient also reported nausea and the other complained of generalized weakness.

The results of this study demonstrated that KADA treatment provided unexpected benefit to patients whose multiple myeloma was refractory to prior treatments. KADA treatment combined treatments with carfilzomib, a proteasome inhibitor; dexamethasone, a glucocorticoid; arsenic trioxide; and ascorbic acid. All of the patients selected for this study had multiple myeloma that was refractory to previous treatments with carfilzomib and a glucocorticoid. Of note, the five patients who achieved a clinical benefit with KADA therapy included patients who were refractory to previous treatments with dexamethasone, arsenic trioxide, or intravenous ascorbic acid as well. Thus, this study demonstrated that the combination of carfilzomib, dexamethasone, arsenic trioxide, and ascorbic acid synergistically benefited the treatment of multiple myeloma, since the combination of these agents could benefit patients whose cancer was refractory to prior, separate treatments with these agents.

### Example 2

### Patient 1027: Previous Carfilzomib-Containing Regimens and KADA Therapy

Patient 1027 had received seven prior lines of treatments for multiple myeloma (IgG kappa) before beginning KADA therapy, including a previous carfilzomib-containing regimen. These treatments took place between Oct. 25, 2011 and Feb. 14, 2014.
Full regimen treatment (5 cycles):
   Carfilzomib on days 1, 2, 8, 9, 15, 16: C1=20 mg/m² IV, C2=27 mg/m² IV, C3=36 mg/m² IV, C4-8=45 mg/m² I
   Dexamethasone 4 mg orally before each carfilzomib treatment
   Ascorbic acid 1000 mg orally on days 1-4
   Melphalan 0.1 mg/kg orally on days 1-4.
Maintenance treatment (18 cycles and 2 days of cycle 19):
   Carfilzomib 36 mg/m² IV on days 1, 2, 15, 16
   Dexamethasone 4 mg IV before each carfilzomib treatment.

The best response from this treatment was a CR that lasted for 17 months. It was determined that the disease was again progressing on Jun. 26, 2013. The date of the last carfilzomib dose under this regimen was Oct. 8, 2013.

Additionally, patient 1027 had previously undergone one arsenic trioxide-containing regimen that took place between Jun. 14, 2005 and Mar. 22, 2006.
Full regimen treatment (6 cycles):
   Arsenic trioxide 0.25 mg/kg IV: C1=on days 1, 2, 3, 4 and then twice weekly, C2-6=twice weekly
   Ascorbic acid 1 g IV after each arsenic trioxide treatment
   Melphalan 0.1 mg/kg orally: C1-6=on days 1-4
Maintenance treatment (6 weeks):
   Arsenic trioxide 0.25 mg/kg IV once weekly
   Ascorbic acid 1 g IV after each arsenic trioxide treatment

The best response from this treatment was a partial response that had six month duration.

Patient 1027 was 73 years old when he began the KADA therapy on Sep. 11, 2013. At the beginning of the treatment, the multiple myeloma met the criteria of stage II according to ISS. An MR was achieved on Sep. 19, 2013 with a 37% decrease in serum M-protein and is ongoing. Patient 1027's lab results during the KADA therapy are summarized in the following table.

TABLE 3

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Sep. 11, 2013 | 1040 | 20 | 19 | 94.9 | 8.2 | 11.57 | 0.67 | NA |
| Sep. 19, 2013 | 790 | 13 | 15 | 83.9 | 5.7 | 14.72 | 0.42 | 124.39 |
| Oct. 2, 2013 | 612 | 11 | 18 | 77 | 1.3 | 59.23 | 0.41 | 170.77 |
| Oct. 30, 2013 | 584 | 8 | 28 | 76.5 | 1.2 | 63.75 | 0.37 | 38.07 |
| Nov. 26, 2013 | NA | NA | NA | NA | NA | NA | 0.38 | NA |
| Dec. 26, 2013 | 563 | 9 | 13 | 81.6 | 1.5 | 54.4 | 0.37 | 107.65 |
| Jan. 22, 2014 | 666 | 9 | 10 | 65 | 4.4 | 14.77 | 0.43 | 131.21 |
| Feb. 19, 2014 | 599 | 9 | 10 | 98.5 | <1.0 | >98.50 | 0.38 | 76.31 |

Patient 1027 had received seven prior treatment regimens before receiving KADA treatment. The patient was refractory to a treatment that included carfilzomib, dexamethasone, and ascorbic acid and to a treatment that included arsenic trioxide. However, the patient received a sustained clinical benefit that is currently ongoing at the time of this submission when these agents were combined for the KADA therapy. This case study demonstrates unexpected, synergistic results of the combination of carfilzomib, dexamethasone, arsenic trioxide, and ascorbic acid, since the combination of all four of these agents could provide the patient with a clinical benefit despite the fact that the patient was previously refractory to other combinations of these agents.

### Example 3

### Patient 1057: Previous Carfilzomib-Containing Regimens and KADA Therapy

Patient 1057 had received five prior lines of treatments for multiple myeloma (lambda light chain disease) before beginning KADA therapy, including a previous carfilzomib-containing regimen. These treatments took place from Mar. 16, 2013 until Dec. 10, 2013.
Full regimen treatment (8 cycles):
   Carfilzomib on days 1, 2, 8, 9, 15, 16: 56 mg/m² IV, reduced on May 21, 2013 to 45 mg/m² IV secondary to fatigue, increased to 56 mg/m² IV on Sep 3, 2013
   Dexamethasone 8 mg IV before each carfilzomib treatment
Maintenance treatment (15 days of cycle 1):
   Carfilzomib 36 mg/m² IV on days 1, 2, 15, 16
   Dexamethasone 4 mg IV before each carfilzomib treatment.

The best response from this treatment was an MR that lasted for 5 months. It was determined that the disease was again progressing on Oct. 22, 2013. The date of the last carfilzomib dose under this regimen was Oct. 8, 2013.

Patient 1057 was 66 years old when she began the KADA therapy on Dec. 7, 2013. At the beginning of the treatment, the multiple myeloma was stage I according to ISS criteria. It was determined that the disease was again progressing on Feb. 7, 2014 and KADA therapy was discontinued. Patient 1057's lab results during the KADA therapy are summarized in the following table.

TABLE 4

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Dec. 10, 2013 | 462 | <7 | 15 | <1.0 | 492.6 | <0.01 | NA | 650.38 |
| Jan. 7, 2014 | 528 | 8 | 15 | <1.0 | 760.7 | <0.01 | NA | 630.12 |
| Jan. 13, 2014 | NA | NA | NA | <1.0 | 819 | <0.01 | NA | NA |
| Jan. 14, 2014 | 492 | 8 | 16 | <1.0 | 457.9 | <0.01 | NA | 634 |
| Feb. 7, 2014 | 473 | <7 | 11 | <1.0 | 607.1 | <0.02 | NA | 1194.79 |

### Example 4

### Patient 1119: Previous Carfilzomib-Containing Regimens and KADA Therapy

Patient 1119 had received six prior lines of treatments for multiple myeloma (IgG lambda disease) before beginning KADA therapy, including a previous carfilzomib-containing regimen. These treatments took place from Dec. 5, 2012 until Jun. 21, 2013.
Full regimen treatment (6 cycles and 22 days of cycle 7):
   Carfilzomib on days 1, 8, 15, 20: C1D1=20 mg/m² IV, otherwise 70 mg/m² IV
   Dexamethasone 40 mg IV before each carfilzomib treatment

The best response from this treatment was an MR that lasted for 5 months. It was determined that the disease was again progressing on Jun. 14, 2013. The date of the last carfilzomib dose under this regimen was Jun. 7, 2013.

Patient 1119 was 44 years old when he began the KADA therapy on Nov. 25, 2013. The multiple myeloma was stage I according to ISS criteria. An MR was achieved on Dec. 17, 2013 with a 31% decrease in serum M-protein from 3.1 g/dL to 2.15 g/dL. The duration of this response was approximately 2 months, at which time the patient progressed and KADA therapy was discontinued on Feb. 18, 2014. Patient 1119's lab results during the KADA therapy are summarized in the following table.

TABLE 5

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Nov. 22, 2013 | 5090 | <7 | <4 | <1.0 | 316.1 | <0.01 | 3.1 | NA |
| Dec. 17, 2013 | 3370 | 8 | <4 | <1.0 | 146.3 | <0.01 | 2.15 | NA |
| Jan. 13, 2014 | 3180 | <7 | <4 | <1.0 | 159.9 | <0.01 | 2.2 | NA |
| Feb. 11, 2014 | 3470 | <7 | <4 | <1.0 | 269.6 | <0.01 | 2.55 | NA |

### Example 5

### Patient 1525: Previous Carfilzomib-Containing Regimens and KADA Therapy

Patient 1525 had received 9 prior lines of treatments for multiple myeloma (kappa light chain disease) before beginning KADA therapy, including a previous carfilzomib-containing regimen that took place from Jan. 24, 2012 until Jul. 30, 2012.
Full regimen treatment (4 cycles and 28 days per cycle):
   Carfilzomib on days 1, 8, 9, 15, 16: C1 days 1, 2: 20 mg/m² IV, otherwise 27 mg/m² IV
   Dexamethasone 4 mg IV before each carfilzomib treatment

The best response from this treatment was PR that lasted for 4 months. It was determined that the disease was again progressing on Oct. 22, 2013. The date of the last carfilzomib dose under this regimen was Oct. 8, 2013.

Patient 1525 was 58 years old when she began the KADA therapy on Aug. 27, 2013. The stage was undetermined. A CR was achieved on Sep. 18, 2013 and is ongoing. Patient 1525's lab results during the KADA therapy are summarized in the following table.

TABLE 6

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Aug. 19, 2013 | 322 | <7 | 18 | 97.8 | 6.7 | 14.60 | NA | NA |
| Sep. 18, 2013 | 280 | <7 | 7 | 19 | <1.0 | >19.00 | NA | NA |
| Oct. 16, 2013 | 276 | <7 | 6 | 16.9 | <1.0 | >16.90 | NA | NA |
| Nov. 12, 2013 | 263 | <7 | 5 | 18.6 | <1.0 | 18.60 | NA | NA |
| Feb. 4, 2014 | 282 | <7 | 7 | 25.9 | <1.0 | >25.90 | NA | NA |

### Example 6

### Patient 1701: Previous Carfilzomib-Containing Regimens and KADA Therapy

Patient 1701 had received seven prior lines of treatments for multiple myeloma (kappa light chain disease) before beginning KADA therapy, including a previous carfilzomib-containing regimen that took place from Jun. 10, 2013 until Jul. 1, 2013.
Full regimen treatment (6 cycles and 2 days of cycle 7) consisted of:
   Carfilzomib on days 1, 2, 8, 9, 15, 16: 40 mg/m² IV
   Dexamethasone on days 1, 8, 22: 40 mg IV, decreased to 20 mg IV on Oct. 22, 2013
   Melphalan 0.1 mg/kg orally on days 1, 2, 3, 4
   Ascorbic acid 1 g orally on days 1, 2, 3, 4
   Clarithromycin 500 mg orally, daily treatment

The best response from this treatment was a PR that lasted for 5 months. However, it was determined that the disease was again progressing on Nov. 18 2013. The date of the last carfilzomib dose under this regimen was Nov. 24 2013.

Patient 1701 was 57 years old when he started the KADA therapy on Dec. 16, 2013. The multiple myeloma was stage III according to ISS criteria. An MR was achieved on Feb. 10, 2014. The duration of the response was approximately one month, at which time the patient was hospitalized secondary to fever of unknown etiology on Mar. 5, 2014. Last administration of KADA was on Mar. 4, 2014. Patient 1701's lab results during the KADA therapy are summarized in the following table.

TABLE 7

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Dec. 9, 2013 | 56 | 861 | 7 | 99.2 | 4.9 | 20.24 | 0.63 | 225.5 |
| Jan. 6, 2014 | 56 | 933 | <4 | 54 | <1.0 | >54.0 | 0.64 | 180.4 |
| Feb. 10, 2014 | 74 | 542 | 17 | 26.3 | <1.0 | >26.30 | 0.36 | 30.8 |

### Example 7

### Patient 1702: Previous Carfilzomib-Containing Regimens and KADA Therapy

Patient 1702 had received ten prior lines of treatments for multiple myeloma (IgG kappa disease) before beginning KADA therapy, including a previous carfilzomib-containing regimen that took place from Jul. 12, 2011 until Mar. 20, 2012.
Full regimen treatment (8 cycles):
   Carfilzomib on days 1, 2, 8, 9, 15, 16: C1=20 mg/m² IV, C2=27 mg/m² IV, C3=36 mg/m² IV, C4-8=45 mg/m² IV
   Dexamethasone 4 mg orally before each carfilzomib treatment
   Cyclophosphamide 200 mg orally on days 1-4
   Ascorbic acid 1,000 mg orally on days 1-4.
Maintenance treatment (15 days of cycle 1):
   Carfilzomib on days 1, 2, 15, 16: 45 mg/m² IV
   Dexamethasone 4 mg IV before each carfilzomib treatment.

The best response from this treatment was a PR that lasted for 5 months. It was determined that the disease was again progressing on March 20, 2012. The date of the last carfilzomib dose under this regimen was Mar. 20, 2012.

Patient 1702 was 57 years old when he began the KADA therapy on Sep. 24, 2013. The multiple myeloma was stage II according to ISS criteria. Though the patient did not meet criteria for progressive disease, serum M-protein and IgG levels increased on Oct. 14, 2013. KADA therapy was discontinued on Oct. 21, 2013. Patient 1702's lab results during the KADA therapy are summarized in the following table.

TABLE 8

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Sep. 16, 2013 | 3460 | 14 | 6 | 6.5 | <1.0 | >6.5 | 4.3 | <34.03 |
| Oct. 14, 2013 | 4210 | 11 | 5 | 6.3 | <1.0 | >6.3 | 4.42 | ND |

### Example 8

### Patient 1795: Previous Carfilzomib-Containing Regimens and KADA Therapy

Patient 1795 had received twelve prior lines of treatments for multiple myeloma (kappa light chain disease) before beginning KADA therapy, including four previous carfilzomib-containing regimens, the first of which took place from Aug. 25th, 2011 until Feb. 28th, 2012.
Full regimen treatment (5 cycles):
   Carfilzomib 1, 2, 8, 9, 15, 16: C1 days 1, 2: 20mg/m² IV, otherwise 27 mg/m² IV
   Dexamethasone 4 mg orally before each carfilzomib treatment

The best response from this treatment was a PR that lasted for 4 months. However, it was determined that the disease was again progressing on Feb. 17, 2012. The date of the last carfilzomib dose under this regimen was Feb. 10, 2012.

Patient 1795 underwent a second carfilzomib-containing regimen from Dec. 11, 2012 until Jan. 30, 2013.
Full regimen treatment (1 cycle and 22 days of cycle 2):
   Carfilzomib on days 1, 2, 8, 9, 15, 16: C1=20 mg/m² IV, C2=27 mg/m² IV
   Dexamethasone on days 1, 8, 15, 22: 40 mg IV
   Clarithromycin XL 1,000 mg orally, daily
   Melphalan 0.1 mg/kg orally on days 1-4
   Ascorbic acid 1 g orally on days 1-4
   Thalidomide 100 mg orally, nightly

The patient progressed on this therapy.

A third carfilzomib-containing regimen took place from Feb. 21, 2013 until Mar. 8, 2013.
Full regimen treatment (16 days of cycle 1) consisted of:
   Carfilzomib on days 1, 2, 8, 9, 15, 16: C1=20 mg/m² IV
   Dexamethasone on days 1, 8, 15, 22: 40 mg IV
   Melphalan 0.1 mg/kg orally on days 1, 2, 3, 4
   Clarithromycin 1,000 mg orally, daily
   Thalidomide 100 mg orally, nightly

The patient progressed on this therapy.

A fourth carfilzomib-containing regimen took place from Apr. 25, 2013 until Aug. 13, 2013.
Full regimen treatment (3 cycles) consisted of:
   Carfilzomib on days 1, 2, 8, 9, 15, 16: 27 mg/m² IV
   Dexamethasone on days 1, 8, 15, 22: 40 mg IV
   Pegylated liposomal doxorubicin on days 1, 2, 8, 15: 6.6 mg/m² IV
   Pomalidomide 2 mg orally on days 1-14

The patient progressed on this therapy.

Patient 1795 was 71 years old when she started the KADA therapy on Sep. 12, 2013. The multiple myeloma was stage II according to ISS criteria. A PR was achieved on Oct. 3, 2013 with a 50% decrease in SFLC. The duration of response was approximately 1 month. KADA therapy was discontinued on Oct. 31, 2013 secondary to tolerability issues, specifically weakness, fatigue and malaise. Patient 1795's lab results during the KADA therapy are summarized in the following table.

TABLE 9

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Sep. 4, 2013 | 49 | <7 | <4 | 9230 | <1.0 | >9230.0 | NA | 2000+ |
| Jan. 6, 2014 | 58 | <7 | <4 | 5870 | <1.0 | >5870.0 | NA | 2000+ |
| Feb. 10, 2014 | 74 | <7 | <4 | 4620 | <1.0 | >4620.0 | NA | NA |

### Example 9

### Patient 2233: Previous Carfilzomib-Containing Regimens and KADA Therapy

Patient 2233 had received seven prior lines of treatments for multiple myeloma (IgG kappa) before beginning KADA therapy, including two previous carfilzomib-containing regimens. A first treatment regimen was administered from Jul. 21, 2012 until May 13, 2013.
Full regimen treatment (8 cycles):
   Carfilzomib on days 1, 2, 8, 9, 15, 16: C1=20 mg/m² IV, C2=27 mg/m² IV, C3=36 mg/m² IV, C4-8=45 mg/m² IV
   Dexamethasone on days 1, 4, 8, 11: 40 mg IV
   Pegylated liposomal doxorubicin on days 1, 4, 8, 11: 4 mg/m² IV
Maintenance treatment (2 cycles and 2 days of cycle 3):
   Carfilzomib on days 1, 2, 15, 16: 45 mg/m² IV, reduced to 36 mg/m² IV upon onset of first cycle of maintenance therapy, and reduced further to 27 mg/m² IV on day 16 of first cycle of maintenance therapy secondary to nausea

The best response from this treatment was a PR that lasted for 9 months. It was determined that the disease was again progressing on May 31, 2013. The date of the last carfilzomib dose under this regimen was May 13, 2013.

Additionally, patient 2233 underwent a second carfilzomib-containing regimen from Oct. 14, 2013 until Oct. 12, 2013.
Full regimen treatment (8 days of 1 cycle):
   Carfilzomib on days 1, 2, 8, 9, 15, 16: 27 mg/m² IV
   Cyclophosphamide 2.2 mg/kg orally
   Ascorbic acid 1 g orally on days 1-4

The patient progressed on this therapy.

Patient 2233 was 54 years old when she started the KADA therapy on Nov. 19, 2013. The multiple myeloma was stage II according to ISS criteria. The patient's serum free light chain and 24-hour urine M-protein improved but did not meet criteria for response so patient achieved SD on Dec. 16, 2013 and the duration of SD was less than one month. KADA therapy was discontinued on Jan. 9, 2014 secondary to tolerability issues, specifically nausea, fatigue and malaise. Patient 2233's lab results during the KADA therapy are summarized in the following table.

TABLE 10

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Nov. 18, 2013 | 323 | 13 | 18 | 6080 | 5.7 | 1066.7 | .3 | 5336.44 |
| Dec. 2, 2013 | NA | NA | NA | 3620 | 5.4 | 670.37 | NA | NA |
| Dec. 16, 2013 | 349 | 11 | 8 | 1110 | <1.0 | >1110.0 | NA | 3903.78 |

### Example 10

### Patient 2417: Previous Carfilzomib-Containing Regimens and KADA Therapy

Patient 2417 had received three prior lines of treatments for multiple myeloma (IgG kappa) before beginning KADA therapy, including a previous carfilzomib-containing regimen that took place Sep. 19, 2013 until Dec. 10, 2013.
Full regimen treatment (5 cycles):
   Carfilzomib on days 1, 2, 8, 9, 15, 16: C1 days 1 and 2=20 mg/m² IV, otherwise 27 mg/m² IV I
   Dexamethasone on days 1, 8, 15, 22: 40 mg IV
   Lenalidomide 10 mg on days 1-14
   Pegylated liposomal doxorubicin on days 1, 8, 15: 5.3 mg/m² IVPB

The best response from this treatment was an SD that lasted for less than one month. It was determined that the disease was again progressing on Dec. 10, 2013. The date of the last carfilzomib dose under this regimen was Nov. 26, 2013.

Patient 2417 was 62 years old when he started the KADA therapy on Dec. 12, 2013. The multiple myeloma was stage II according to ISS criteria. The patient progressed and KADA therapy was discontinued on Jan. 27, 2014. Patient 2417's lab results during the KADA therapy are summarized in the following table.

TABLE 11

| | | | | | | | | Urine 24 hr |
|---|---|---|---|---|---|---|---|---|
| | | | | | | | Serum M- | M- |
| | IgG | IgA | IgM | Kappa | Lambda | Kappa/ | protein | protein |
| Date | (mg/dL) | (mg/dL) | (mg/dL) | (mg/L) | (mg/L) | Lambda | (g/dL) | (mg) |
| Dec. 10, 2013 | 291 | 8 | 26 | 319.4 | 5 | 63.88 | NA | <25.14 |
| Jan. 6, 2014 | 308 | <7 | 18 | 376 | <1.0 | >376.0 | NA | ND |

### Example 11

### Criteria for Multiple Myeloma Stages and Responses

### International Staging System

The International Staging System (ISS) for myeloma was published by the International Myeloma Working Group:
Stage I: 32-microglobulin ((32M)<3.5 mg/L, albumin≧3.5 g/dL
Stage II: 32M<3.5 mg/L and albumin<3.5 g/dL; or β2M 3.5 mg/L-5.5 mg/L irrespective of the serum albumin
Stage III: β2M≧5.5 mg/L

Source: Greipp P R, San Miguel J, Durie B G, et al., International staging system for multiple myeloma. J Clin Oncol. 2005; 23(15):3412-20.

### Uniform Response Criteria for Multiple Myeloma

The standards of the International Myeloma Working Group Uniform Response Criteria (IMWG) are summarized in the following table.

TABLE 12

| Response | IMGW |
|---|---|
| Subcategory | Criteria |
| sCR: | CR as defined below plus normal FLC ratio and absence of clonal cells in |
| stringent | bone marrow3 by immunohistochemistry or immunofluorescence ² |
| complete | |
| response | |
| CR: | Negative immunofixation on the serum and urine and disappearance of any |
| complete | soft tissue plasmacytomas and <5% plasma cells in bone marrow ¹ |
| response | |
| VGPR: | Serum and urine M-protein detectable by immunofixation but not on |
| Very good | electrophoresis or ≧90% reduction in serum M-protein plus urine M- |
| partial | protein level <100 mg/24 h |
| response | |
| PR: | ≧50% reduction of serum M-protein and reduction in 24 hours urinary |
| partial | M-protein by ≧90% or to <200 mg/24 h |
| response | If the serum and urine M-protein are unmeasurable, ³ a ≧50% decrease in |
| | the difference between involved and uninvolved FLC levels is required in |
| | place of the M-protein criteria |
| | If serum and urine M-protein are not measurable, and serum free light |
| | assay is also not measureable, ≧50% reduction in plasma cells is |
| | required in place of M-protein, provided baseline bone marrow plasma |
| | cell percentage was ≧30% |
| | In addition to the above listed criteria, if present at baseline, a ≧50% |
| | reduction in the size of soft tissue plasmacytomas is also required |
| SD: | Not meeting criteria for CR, VGPR, PR, or progressive disease |
| stable | |
| disease | |
| PD: | Increase of ≧25% from lowest response value in any one or more of the |
| partial | following: |
| disease | Serum M-component and/or (the absolute increase must be ≧200 |
| | mg/24 h)⁴ |
| | Urine M-component and/or (the absolute increase must be |
| | 200 mg/24 h) |
| | Only in patients without measurable serum and urine M-protein levels; |
| | the difference between involved and uninvolved FLC levels. The |
| | absolute increase must be >10 mg/dL |
| | Bone marrow plasma cell percentage; the absolute percentage must be |
| | ≧10%⁵ |
| | Definite development of new bone lesions or soft tissue plasmacytomas |
| | or definite increase in the size of existing bone lesions or soft tissue |
| | plasmacytomas. |
| | Development of hypercalcaemia (corrected serum calcium >11.5 |
| | mg/dL or 2.65 mmol/L) that can be attributed solely to the plasma cell |
| | proliferative disorder. |

A clarification to IMWG criteria for coding CR and VGPR in patients in whom the only measurable disease is by serum FLC levels: CR in such patients is defined as a normal FLC ratio of 0.26-1.65 in addition to CR criteria listed above. VGPR in such patients is defined as a >90% decrease in the difference between involved and uninvolved free light chain (FLC) levels.
¹ Confirmation with repeat bone marrow biopsy not needed.
² Presence/absence of clonal cells is based upon the kappa/lambda ratio. An abnormal kappa/lambda ratio by immunohistochemistry and/or immunofluorescence requires a minimum of 100 plasma cells for analysis. An abnormal ratio reflecting presence of an abnormal clone is kappa/lambda of >4:1 or <1:2.
³All relapse categories require two consecutive assessments made at anytime before classification as relapse or disease progression and/or the institution of any new therapy. In the IMWG criteria, CR patients must also meet the criteria for progressive disease shown here to be classified as progressive disease for the purposes of calculating time to progression and progression-free survival. The definitions of relapse, clinical relapse and relapse from CR are not to be used in calculation of time to progression or progression-free survival.
⁴For progressive disease, serum M-component increases of >1 gm/dL are sufficient to define relapse if starting M-component is >5 g/dL.
⁵Relapse from CR has the 5% cut-off versus 10% for other categories of relapse.
⁶ For purposes of calculating time to progression and progression-free survival, CR patients should also be evaluated using criteria listed above for progressive disease.

Source: B G M Durie et al., International uniform response criteria for multiple myeloma. Leukemia (2006) 1-7. Adapted from Durie B G M, et al., Leukemia 2006; 20: 1467-1473; and Kyle R A, Rajkumar S V. Leukemia 2008;23:3-9.

TABLE 13
Definition of the Minimal Response per EMBT Criteria

| Response | |
|---|---|
| Subcategory | Multiple Myeloma Response Criteria |
| MR: | 25% to 49% reduction in the level of serum |
| minimal | M-protein or a 50% to 89% reduction in |
| response | 24-hour urinary M-protein, which still |
| | exceeds 200 mg/24 hr, maintained for a |
| | minimum of 8 weeks.¹ |
| | 25% to 49% reduction in the size of soft- |
| | tissue plasmacytomas (by radiography or |
| | clinical examination) |

¹EBMT response criteria updated from 6 to 8 weeks to coincide with scheduled disease assessments timing
Abbreviations: EBMT = European Group for Blood and Marrow Transplantation; hr = hour(s).

### SOURCES

Bladé J, Samson D, Reece D, et al., European Group for Blood and Marrow Transplant. Myeloma Subcommittee of the EBMT. Criteria for evaluating disease response and progression in subjects with multiple myeloma treated by high-dose therapy and hematopoietic stem cell transplantation. Br J Haematol. 1998; 102(5):1115-23.

Kyle R A, Rajkumar S V. Criteria for diagnosis, staging, risk stratification and response assessment of multiple myeloma: Criteria for assessment of multiple myeloma. Leukemia. 2009; 23: 3-9.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating or preventing a hematological malignancy in a subject comprising administering to the subject a proteasome inhibitor, a glucocorticoid, an arsenic-containing compound, and ascorbic acid or a derivative thereof.

2. The method of claim 1, wherein the hematological malignancy is selected from the group consisting of: multiple myeloma, chronic lymphocytic leukemia, or B-cell non-Hodgkin lymphoma.

3. The method of claim 1, wherein the proteasome inhibitor is selected from the group consisting of: bortezomib, carfilzomib, oprozomib, ixazomib citrate, marizomib, delanzomib, and syringolin A.

4. The method of claim 1, wherein the proteasome inhibitor is carfilzomib.

5. The method of claim 1, wherein the glucocorticoid is selected from the group consisting of: hydroxycortisone, cortisone, desoxycorticosterone, fludrocortisone, betamethasome, dexamethasone, prednisolone, prednisone,
methylprednisolone, methylprednisone, paramethasone, triamcinolone, flumethasone, fluocinolone, fluocinonide, fluprednisolone, halcinonide, flurandrenolide, meprednisone, and medrysone.

6. The method of claim 1, wherein the glucocorticoid is dexamethasone.

7. The method of claim 1, wherein the arsenic-containing compound is selected from the group consisting of: arsenic trioxide (As203), arsenic pentoxide (As205), arsenic hexoxide As406), arsenic triselenide (As2Se3), arsenic disulfide (As2S2), arsenic trisulfide (As2S3), arsenic pentasulfide (As205), arsenic tritelluride (As2Te3), sodium arsenate (Na2HAs04), potassium arsenate (KH2As04), and sodium arsenyl tartrate (NaC4H4As06).

8. The method of claim 1, wherein the arsenic-containing compound is arsenic trioxide.

9. The method of claim 1, wherein the ascorbic acid or derivative thereof is selected from the group consisting of: ascorbic acid, L-ascorbic acid-2-pyrophosphate esters, L-ascorbic acid-2-triphosphate esters, L-ascorbic acid-2-polyphosphate esters, sodium L-ascorbic acid-2-phosphate-6-palmitate, L-ascorbic acid-2-phosphate-6-palmitate, L-ascorbic acid-2-phosphate-6-stearate, L-ascorbic acid-2-phosphate-6-oleate and L-ascorbic acid-2-phosphate-6-arachidonate, 5,6-0-isoalkylidene ascorbic acid, 5,6-0-isopropylidine ascorbic acid, and L-ascorbate 2-sulphate.

10. The method of claim 1, wherein the ascorbic acid or derivative thereof is ascorbic acid.

11. The method of claim 1, comprising administering carfilzomib, arsenic trioxide, dexamethasone, and ascorbic acid.

12. The method of claim 1, comprising administering carfilzomib intravenously.

13. The method of claim 1, comprising administering carfilzomib at a dose of 1-100 mg/m².

14. The method of claim 1, comprising administering arsenic trioxide intravenously.

15. The method of claim 1, comprising administering arsenic trioxide at a dose of 0-5 mg/kg.

16. The method of claim 1, comprising administering dexamethasone orally.

17. The method of claim 1, comprising administering dexamethasone intravenously.

18. The method of claim 1, comprising administering dexamethasone at a dose of 1-100 mg.

19. The method of claim 1, comprising administering ascorbic acid orally.

20. The method of claim 1, comprising administering ascorbic acid orally at a dose of 100-2000 mg.

21. The method of claim 1, comprising administering ascorbic acid intravenously.

22. The method of claim 1, comprising administering ascorbic acid intravenously at a dose of 1-50 mg.

23. A method of treating or preventing multiple myeloma in a subject comprising administering to the subject a proteasome inhibitor, arsenic trioxide, a glucocorticoid, and ascorbic acid.

24.-43. (canceled)

44. A method of treating or preventing a relapsed hematological malignancy in a subject comprising administering to the subject, a glucocorticoid, an arsenic-containing compound, and ascorbic acid or a derivative thereof.

45. A method of treating or preventing hematological malignancy that is refractory to a prior treatment or treatments for cancer in a subject comprising administering to the subject a proteasome inhibitor, a glucocorticoid, an arsenic-containing compound, and ascorbic acid or a derivative thereof.

46-71. (canceled)
